(19) United States
(12) Patent Application Publication
(10) Pub. No.: US 2007/0020334 A1
(43) Pub. Date: Jan. 25, 2007

(54) Benzimidazole formulation

(76) Inventors: Poul Bertelsen, Roskilde (DK); Peder Mohr Olsen, Kirke Hyllinge (DK)

Correspondence Address: WENDEROTH, LIND & PONACK, L.L.P., 2033 K STREET N. W., SUITE 800, WASHINGTON, DC, 20006-1021, US

(21) Appl. No.: 11/483,736

(22) Filed: Jul. 11, 2006

Related U.S. Application Data
(60) Provisional application No. 60/727,855, filed on Oct. 19, 2005.

(30) Foreign Application Priority Data
Jul. 11, 2005 (DK) PA 2005 01019

(51) Int. Cl. A61K 31/4439; A61K 31/4184; A61K 9/24; B27N 3/00

(52) U.S. Cl. 424/472; 514/338; 514/394; 264/109

(57) ABSTRACT

A dry manufacturing process for the production of a pharmaceutical formulation of a benzimidazole and an alkaline substance is described. A tablet is compressed directly from a dry powder or a dry particulate matter avoiding any liquid or excipient conventionally used as a wet binder. The manufacturing process has the advantage of being simple and cost efficient. At the same time an expensive drying step is superfluous. The resulting pharmaceutical formulation has a good stability and a good dissolution profile.

The present application claims the benefit of U.S. provisional application Ser. No. 60/727,855, filed Oct. 19, 2005.

### FIELD OF INVENTION

The present invention relates to the field of pharmaceutical formulation science. In particular, the present invention relates to pharmaceutical formulations comprising acid labile benzimidazoles. The invention provides cost-effective production methods providing stable formulations.

### BACKGROUND

Due to the acid labile nature of the benzimidazoles it is necessary to protect the drug substance from exposure to acids, especially in the presence of humidity. In the first stage, the benzimidazole has to be protected from acid attack during storage of the drug. In the prior art, this is provided by contacting the benzimidazole with an alkaline substance present in the pharmaceutical formulation. In the next stage the benzimidazole must be protected from acid attack in the stomach. The person skilled in the art will realise that this can be achieved by applying an enteric coating. The active drug, the alkaline substance and pharmaceutical excipients are usually formulated in laborious approaches employing wet granulation.

There is thus a need in the art for simple and cost efficient manufacturing methods for producing benzimidazole formulations with good stability properties and good dissolution profiles. There is furthermore a need in the art for obtaining such tablets in a relatively small size for efficient passage and drug delivery in the intestinal tract.

### SUMMARY OF INVENTION

The object of the present invention thus to provide a stable and cost-efficient pharmaceutical formulation intended for oral administration and subsequent efficient delivery of the active benzidimazole in the intestinal tract, wherein said formulation has a good shelf life stability and release profile.

In particular, the present invention relates to a method for producing a pharmaceutical tablet formulation comprising a benzimidazole as the biologically active component, wherein:
said formulation comprises an enteric coating for protection of the active component from acid attack in the stomach,
said benzimidazole is further stabilized by an alkaline substance in the tablet,
said method comprising dry granulating steps and dry compressing of tablets, wherein said formulation is further characterized by one or more of the following features:
   (i) the alkaline substance is an alkali metal carbonate with high water solubility and a BET area of at least about 1 m²/g prior to any dry granulation and/or dry compression steps,
   (ii) the alkaline substance is an alkaline earth metal carbonate with low water solubility and a BET area of at least about 1 m²/g prior to any dry granulation and/or dry compression steps,
   (iii) the benzimidazole and the alkaline substance have been mixed and dry granulated together prior to dry compression,
   (iv) the weight ratio of benzimidazole and alkaline substance is from about 1:0.2-1:5,
   (v) the alkaline substance has a pKa of at least about 10 and a BET area of at least about 1 m²/g prior to any dry granulation and/or dry compression steps,
   (vi) if the alkaline substance is polyvalent, said alkaline substance has a pKa1-value of 6 or more and a BET area of at least about 1 m²/g prior to any dry granulation and/or dry compression steps,
   (vii) the alkaline substance has a BET-area of at least about 1 m²/g prior to any dry granulation and/or dry compression steps,
   (viii) the tablet formulation further comprises a disintegrant in an amount of about 1-30% by weight.

The invention furthermore relates to a pharmaceutical tablet formulation comprising a benzimidazole as the biologically active component, wherein:
said formulation comprises an enteric coating for protection of the active component from acid attack in the stomach,
said benzimidazole is further stabilized by an alkaline substance in the tablet, wherein said formulation is further characterized by one or more of the following features:
   (i) the alkaline substance raw material is an alkali metal carbonate with high water solubility and a BET area of at least about 1 m²/g,
   (ii) the alkaline substance raw material is an alkaline earth metal carbonate with low water solubility and a BET area of at least about 1 m²/g,
   (iii) the benzimidazole and the alkaline substance raw material have been mixed and dry granulated together prior to dry compression,
   (iv) the weight ratio of benzimidazole and alkaline substance is from about 1:0.2-1:5,
   (v) the alkaline substance raw material has a pKa of at least about 10 and a BET area of at least about 1 m²/g,
   (vi) if the alkaline substance is polyvalent, said alkaline substance has a pKa1-value of 6 or more and a BET area of at least about 1 m²/g,
   (vii) the alkaline substance raw material has a BET-area of at least about 1 m²/g,
   (viii) the tablet formulation further comprises a disintegrant in an amount of about 1-30% by weight.

The manufacturing process involves only few production steps and the use of any liquid is avoided rendering an expensive drying step superfluous. A liquid can, however, be applicable in the subsequent coating steps providing an enteric coat and optionally a subcoat.

### DETAILED DESCRIPTION OF THE INVENTION

In a first aspect, the present invention relates to a pharmaceutical tablet formulation comprising a benzimidazole as the biologically active component, wherein:
said formulation comprises an enteric coating for protection of the active component from acid attack in the stomach,
said benzimidazole is further stabilized by an alkaline substance in the tablet, wherein said formulation is further characterized by one or more of the following features:
   (i) the alkaline substance raw material is an alkali metal carbonate with high water solubility and a BET area of at least about 1 m²/g,
   (ii) the alkaline substance raw material is an alkaline earth metal carbonate with low water solubility and a BET area of at least about 1 m²/g,
   (iii) the benzimidazole and the alkaline substance raw material have been mixed and dry granulated together prior to dry compression,
   (iv) the weight ratio of benzimidazole and alkaline substance is from about 1:0.2-1:5,
   (v) the alkaline substance raw material has a pKa of at least about 10 and a BET area of at least about 1 m²/g,
   (vi) if the alkaline substance is polyvalent, said alkaline substance has a pKa1-value of 6 or more and a BET area of at least about 1 m²/g,
   (vii) the alkaline substance raw material has a BET-area of at least about 1 m²/g,
   (viii) the tablet formulation further comprises a disintegrant in an amount of about 1-30% by weight.

In a second aspect, the present invention relates to a method for producing a pharmaceutical tablet formulation comprising a benzimidazole as the biologically active component, wherein:
said formulation comprises an enteric coating for protection of the active component from acid attack in the stomach,
said benzimidazole is further stabilized by an alkaline substance in the tablet,
said method comprising dry granulating steps and dry compressing of tablets wherein said formulation is further characterized by one or more of the following features:
   (i) the alkaline substance is an alkali metal carbonate with high water solubility and a BET area of at least about 1 m²/g prior to any dry granulation and/or dry compression steps,
   (ii) the alkaline substance is an alkaline earth metal carbonate with low water solubility and a BET area of at least about 1 m²/g prior to any dry granulation and/or dry compression steps,
   (iii) the benzimidazole and the alkaline substance have been mixed and dry granulated together prior to dry compression,
   (iv) the weight ratio of benzimidazole and alkaline substance is from about 1:0.2-1:5,
   (v) the alkaline substance has a pKa of at least about 10 and a BET area of at least about 1 m²/g prior to any dry granulation and/or dry compression steps,
   (vi) if the alkaline substance is polyvalent, said alkaline substance has a pKa1-value of 6 or more and a BET area of at least about 1 m²/g prior to any dry granulation and/or dry compression steps,
   (vii) the alkaline substance has a BET-area of at least about 1 m²/g prior to any dry granulation and/or dry compression steps,
   (viii) the tablet formulation further comprises a disintegrant in an amount of about 1-30% by weight.

In a preferred embodiment the benzimidazole is pantoprazole such as pantoprazole sodium hydrate or pantoprazole sodium sesquihydrate. In other preferred embodiments the benzimidazole may be omeprazole or a salt and/or a hydrate thereof, lansoprazole or a salt and/or a hydrate thereof, esomeprazol or a salt and/or a hydrate thereof, aripiprazole or a salt and/or a hydrate thereof, rabeprazol or a salt and/or a hydrate thereof, or timoprazole or a salt and/or a hydrate thereof.

In another preferred embodiment, said formulation further comprises other pharmaceutically acceptable excipients such as fillers, dry binders, glidants and lubricants. In a particularly preferred embodiment, said formulation comprises crospovidone as a disintegrant in an amount of from about 5-20%, preferably 7.5-15%, most preferably 10-13% by weight.

In yet another embodiment, said formulation comprises a subcoat. In a particularly preferred embodiment however, said formulation does not comprise a subcoat. In the Examples it is shown that it is possible according to the present invention to obtain tablet formulations with good stability properties and good dissolution profiles without the laborious steps of applying a subcoat.

In yet another preferred embodiment, the alkaline substance is a salt of an organic or an inorganic acid where the anion of the salt is carbonate (CO₃²⁻), hydrogenphosphate (HPO₄²⁻) or phosphate (PO₄³⁻). The alkaline substance may also be a salt of an organic or an inorganic acid where the kation is sodium (Na⁺), calcium (Ca²⁺) or magnesium (Mg²⁺). Preferably, the salt of the organic and/or inorganic acid according is sodiumcarbonate (Na₂CO₃), or Calciumcarbonate (CaCO₃) trisodiumphosphate (Na₃PO₄), disodiumhydrogenphosphate (Na₂HPO₄), hydrazine or derivatives thereof, lysine or a derivative thereof, arginine or a derivative thereof, or histidine or a derivative thereof.

In yet another preferred embodiment, dry granulation is provided by means of a roller compactor.

In a final preferred embodiment, the mixture has been subject to sieving prior to tablet compression with a sieve size (Roller compactor) of 1.25 mm or less. It is shown in the examples that relatively small and relatively homogenous particles result in a more accurate dosing of the active compound. Doze accuracy is of particular importance in production of relatively small tablets.

In a final aspect, the present invention relates to products obtainable or obtained by the methods disclosed herein.

Definitions

Pharmaceutical Tablet Formulation:

A pharmaceutical tablet formulation according to the present invention is equivalent to a solid dosis form.

Drugs:

According to the present invention, the drug substance belongs to the group of benzimidazoles or salts and/or hydrates thereof. The benzimidazole is preferably pantoprazole, omeprazole, lansoprazole, timoprazol, aripiprazole, rabeprazol or esomeprazole, as well as pharmaceutically acceptable salts, hydrates and mixtures thereof. Preferably, the benzimidazole is pantoprazole sodium sesquihydrate. Any pharmaceutically acceptable salt can be used. Examples of conventionally used salts are sodium or potassium salts of the drug substance.

A pharmaceutical formulation according to the present invention comprises about 1 to 500 mg drug pr. dose; such as 1 to 200 mg; or 1 to 100 mg. Preferably, the unit dose comprises 10-120 mg; 15-100 mg; 15-80 mg, 15-70 mg; 15-60 mg; 15-50 mg; mg; such as 20, 30, or 40 mg of benzimidazole, preferably pantoprazole.

Unit dose is a pharmaceutically formulated unit comprising the dosage of drug substance intended for administration. The dosage unit can be a tablet.

Alkaline Substance:

Due to the acid labile nature of the drug substance, the unit dose comprises an alkaline substance, or a mixture of two or more different alkaline substances, in the core to confer shelf life stability of the pharmaceutical formulation.

The alkaline substance according to the present invention may be soluble in water or even practically insoluble in water. E.g., 1 part of water soluble alkaline substance might be dissolved in about e.g. 100, 50, 30, or 10 parts of water or less. 1 part of alkaline substance with low water solubility may be dissolved in at least about 100, 300, 500, 1000, 10,000 or even more than 10,000 parts of water. This is in contrast to conventional production methods employing wet granulation wherein water soluble alkaline substances are preferred. The rationale behind using water soluble alkaline substances in conventional methods is that water soluble alkaline substances are thought to generate a humid environment with an alkaline pH protecting the active drug substance during disintegration of the tablet in the gastric system. In the present invention it is surprisingly demonstrated in the Examples 6, 8 og 15 that alkaline substances such as calcium carbonate which are practically insoluble in water (1 part in more than 10,000 parts of water according to handbook of Pharmaceutical Excipients, 5^{th} ed.) may result in stable formulations with good dissolution profiles. It however appears that alkaline substances with low water solubility should preferably have a relatively large BET area (about 1 m²/g or more).

Generally, alkaline earth metal salts (such as e.g. calcium carbonate, magnesium oxide, magnesium carbonate) tend to have low water solubility. On the other hand, alkali metal salts (such as e.g. sodium carbonate and potassium carbonate) tend to be more water soluble.

According to the prior art such as e.g. WO05009410 (Dr. Reddy's), ratios between benzimidazole and alkaline substance of about 1:0.17 are disclosed (Examples 1 and 2). On basis of the existing knowledge in the art, the skilled man would thus not expect that it would be possible to use ratios of 1:0.2 and above and definitely not ratios of about 1:0.5 or 1:1 or more since increasing amounts of base would be expected to result in slow dissolution profiles of the active compound. According to the present invention however, the weight ratio between the drug substance and the alkaline substance ranges between while surprisingly still providing formulations with a combination of good shelf life stabilities and good dissolution profiles (examples 6, 8, and 15). The weight ratio between the drug and the alkaline substance may thus be about 1:0.2, or 1:0.3, or 1:0.4, or 1:0.5, or 1:0.6, or 1: 0.7, or 1: 0.8, or 1:0.9 or 1:1, or 1:1.5, or 1:2, or 1:2.5, or 1:3, or 1:3.5, or 1:4, or 1:4.5, or 1:5, or 1:6, or 1:7, or 1:8, or 1:9, or 1:10.

It is preferred that the pKa of the chosen alkaline material is at least 10. However, this alone is not sufficient. If the alkaline material is polyvalent the pKaI, (where pKaI is the most acidic pKa value) should be above 6. As shown in ex. 6 the use of tricalcium phosphate which has a pKaI of 2.2 results in a poorer stability than if disodium carbonate (which has a pKaI of 6.4) is used.

Alkaline substance preferably have a pKa value of 6 or above. The alkaline substance will typically provide an alkaline pH in the range of 7-12, when being dissolved and/or dispersed in water at room temperature in an amount of about 10-100 mg/ml.

Accordingly, the term alkaline substance includes the corresponding base of an organic or an inorganic acid, such as provided in the form of a pharmaceutically acceptable salt of an organic or inorganic acid and/or a mixture thereof, and some amino acids. According to the present invention, it is understood that a pharmaceutical formulation may very well comprise more than one alkaline substance, if appropriate.

The alkaline substance raw material is understood to be the alkaline substance prior to any formulation processing steps.

Examples of alkaline substances are listed in the following table.

TABLE 1
Examples of alkaline substances

| Substance | Examples | Structure | pKa* |
|---|---|---|---|
| Salt of carbonic acid | DiSodium carbonate | Na₂CO₃ | 10.3 |
| (carbonate) and | Sodium hydrogen | NaHCO₃ | 6.4 |
| phosphoric acid | carbonate | | |
| (phosphate). Soluble | Trisodium | Na₃PO₄ | 12.4 |
| salts with pKa- | phosphate | | |
| values of 9 and | Disodium hydrogen | Na₂HPO₄ | 7.2 |
| above | phosphate | | |
| | Sodium dihydrogen | NaH₂PO₄ | 2.2 |
| | phosphate | | |
| Salt of carbonic acid | Calcium carbonate | CaCO₃ | 10.3 |
| (carbonate). | | | |
| Practically insoluble | | | |
| with pKa-value | | | |
| of 9 or above | | | |
| Amino acids with | Lysine | C₆H₁₄O₂N₂ | pKa₂: 8.9 |
| pKa₃-Values of | | | pKa₃: 10.3 |
| 9 or above | Arginine | C₆H₁₄N₄O₂ | pKa₂: 9.1 |
| | | | pKa₃: 13.2 |
| | | | pH≈11.4 |
| | | | (100 g/L H₂O) |
| | Histidine | C₆H₉O₂N₃ | pKa₃: 9.0 |
| | | | pH≈7.7 |
| | | | (10 g/L H₂O) |

*The pKa-values in this table are approximate values and refer to the pKa of the acid. Only relevant pKa values are included.

A suitable disintegration time means that the pharmaceutical formulation must comply with the standards set up in the European Pharmacopoeia. Those skilled in the art will appreciate that it is desirable for compressed tablets to disintegrate within 30 minutes, most desirable within 15 minutes upon contact with an aqueous solution, provided that the enteric coating is absent or bursted. Disintegration is preferably performed in a dissolution apparatus such as the Ph. Eur. Basket method as disclosed in e.g. example 11.

Furthermore, it should be understood that the alkaline substance should be provided in solid form, such as in the form of a powder, granulate or the like.

In connection with the present invention (example 16) it has been demonstrated that different alkaline substances may have different surface areas (BET areas) and that the same compound purchased under different trade names (e.g. calcium carbonate—“Sturcal L” and “Scoralite”) may have different BET areas (see the SEM pictures in the figures). It is furthermore demonstrated that alkaline substances with relatively large BET areas (at least about 0.5, 0.6, 0.7, 0.8, 0.9, preferably at least about 1.0, 1.1, 1.2, 1.3, 1.4, and most preferably 1.5 m²/g or more) tend to result in tablets with improved stability properties while at the same time retaining good dissolution properties (example 15). A plausible explanation for this finding is that porous alkaline substances with relatively large BET areas used as a raw material tend to be crushed into fine particles upon mechanical pressure such as e.g. dry granulation and/or dry compression. In contrast, substances with relatively small BET areas (such as e.g. calcium carbonate purchased under the trade name “Scoralite”) tends to either not being affected by mechanical pressure and/or to be crushed into relatively large particles and/or to show a slightly improved distribution of the particles around the drug substance. It is thus preferred to use porous and/or polycrystallic alkaline substances with relative large BET area having a tendency to be crushed into very fine particles upon mechanical pressure. Such alkaline substances in the form of very fine particles in the resulting tablet most likely provide a better “alkaline shield” against acid and humidity attacks of the active compound by providing a close physical contact between the drug and the protective alkaline substance. It is shown in Example 10 that a particularly preferred way of providing a close physical contact and thus a stable tablet with good dissolution profiles is to include a step wherein the two substances are mixed and subsequently dry granulated together prior to compression into a tablet.

Pharmaceutical Excipients:

It is shown in the Examples that it is possible to compress tablets with good stability and dissolution profiles where the only pharmaceutical excipient, apart from the alkaline substance, is minute amounts of MgStearate (example 5). When manufacturing tablets according to the invention in larger scale, such as in production scale, it can however be advantageous to add at least one of the following ingredients: a glidant, a lubricant, a filler, a dry binder, a color, and a disintegrant to the formulation comprising alkaline substance and a benzimidazole. Preferably, the only additional pharmaceutical excipient is a glidant or a lubricant, preferably along with at least one disintegrant, thus providing a simple and cost efficient production method.

Examples of glidant and lubricants are stearic acid, metallic stearates, talc, colloidal silica, sodium stearyl fumarate and alkyl sulphates.

In the present invention, a dry binder such as e.g. sorbitol, isomalt, or mixtures thereof may be used. The dry binder provides the effect of binding a material and thereby providing a powder that can be compressed into a tablet.

A filler substance is any pharmaceutically acceptable substance that does not interact with the drug substance or with other excipients. Commonly used filler substances are: mannitol, Dextrins, maltodextrins (e.g. Lodex® 5 and Lodex® 10), inositol, erythritol, isomalt, lactitol, maltitol, mannitol, xylitol, low-substituted hydroxypropylcellulose (e.g LH 11, LH 20, LH 21, LH 22, LH 30, LH 31, LH 32 available from Shin-Etsu Chemical Co.), starches or modified starches (e.g potato starch, maize starch, rice starch, pre-gelatinised starch), polyvinylpyrrolidone, polyvinylpyrrolidone/vinyl acetate copolymer, agar (e.g. sodium alginate), carboxyalkylcellulose, dextrates, gelatine, gummi arabicum, hydroxypropyl cellulose, hydroxypropylmethylcellulose, methylcellulose, polyethylene glycol, polyethylene oxide, polysaccharides e.g. dextran, soy polysaccharide, sodium carbonate, and sodium chloride.

A wet binder is an excipient that in combination with water facilitates a powder to be compressed into coherent bodies such as tablets or facilitates a powder to be granulated into a particulate matter. A wet binder must, at least to some extent, be soluble in water. Examples of wet binders are PVP (polyvinylpyrrolidone), HPMC (hydroxymethylpropylcellulose) or gelatine. If a wet binder is used according to the present invention, the wet binder will merely act as a filler and will not exhibit the binding properties normally associated with such wet binders. It will therefore be understood that excipients conventionally regarded as wet binders might be used as mere fillers in the context of the present invention.

A disintegrant is a pharmaceutically acceptable substance that improves the disintegration of tablets without interacting with the drug substance or with any other excipients. The disintegrant has the capability of swelling upon contact with water, causing the tablet to swell/disintegrate and thus releasing the active compound. This effect is shown in the Examples (example 18), where dissolution profiles are improved upon addition of disintegrant in the tablet. Traditional wet granulated benzimidazole formulations normally comprise large amounts of disintegrants (at least about 30%) since “wet” production steps cause a significant proportion of the disintegrant to swell and thus irreversibly reducing its swelling capacity. However, in connection with the present invention it has surprisingly been shown that it is possible to obtain a good dissolution profile using relatively small amounts of disintegrant thus enabling production of smaller tablets using more cost-efficient methods (disintegrants are often relatively expensive ingredients). Small tablets have the advantage of being easier to swallow, pack, store, transport, etc. Small tablets furthermore improve movement through the gastric system and are less dependent on the gastric emptying. This effect is probably achieved because dry granulation techniques do not result in unwanted preswelling of the disintegrant. It is furthermore shown that it is possible to use water in connection with the subcoating and/or the enteric coating process without causing unwanted preswelling and/or disintegration of the tablets (example 12, FIG. 8).

Examples of commonly used disintegrants are: Alginic acid—alginates, carboxymethylcellulose calcium, carboxymethylcellulose sodium, crospovidone, hydroxypropylcellulose, hydroxypropylmethylcellulose (HPMC), cellulose derivatives such as low-substituted hydroxypropylcellulose (e.g LH 11, LH 20, LH 21, LH 22, LH 30, LH 31, LH 32 available from Shin-Etsu Chemical Co.) and microcrystalline cellulose, polacrilin potassium or sodium, polyacrylic acid, polycarbofil, polyethylene glycol, polyvinylacetate, crosslinked polyvinylpyrrolidone (e.g. Polyvidon® CL, Polyvidon® CL-M, Kollidon® CL, Polyplasdone® XL, Polyplasdone® XL-10); sodium carboxymethyl starch (e.g. Primogel® and Explotab®), sodium croscarmellose (i.e. cross-linked carboxymethylcellulose sodium salt; e.g. Ac-Di-Sol®), sodium starch glycolate, starches (e.g potato starch, maize starch, rice starch), and pre-gelatinised starch.

The disintegrant may be present in the tablet in an amount of about 1-30%, preferably 3-25%, more preferably 5-20% and most preferably 10-15%, and even most preferably about 8-14%.

Granulation and Compression:

Powders comprising either the drug in question, the alkaline substance, the pharmaceutical excipient(-s), or any combination thereof are subjected to a dry granulation process. The dry granulation process causes the powder to agglomerate into larger particles having a size suitable for further processing. Dry granulation can thus be said to improve the flowability of a mixture in order to be able to produce tablets that comply with the demand of mass variation or content uniformity set out in the European Pharmacopoeia.

Formulations according to the invention may be produced using one or more mixing and dry granulations steps. The order and the number of the mixing and granulation steps do not seem to be critical. However, it seems to be of importance that at least one of the alkaline substance and the drug has been subject to dry granulation before compression into tablets. Dry granulation of drug and alkaline substance together prior to tablet compression seem, surprisingly, to be a simple, inexpensive and efficient way of providing close physical contact between the alkaline substance and the drug and thus a tablet formulation with good stability properties. Relatively large BET areas of the alkaline raw material do also have a beneficial effect on the stability properties.

Dry granulation is carried out by a mechanical process, which transfers energy to the mixture without any use of any liquid substances (neither in the form of aqueous solutions, solutions based on organic solutes, or mixtures thereof) in contrast to conventional wet granulation processes. Generally, the mechanical process requires compaction such as the one provided by roller compaction. An example of an alternative method for dry granulation is slugging.

Roller compaction is a process comprising highly intensive mechanical compacting of one or more substances. The powder is pressed, that is roller compacted, between 2 counter rotating rollers to make a solid sheet which is subsequently crushed in a sieve to form a particulate matter. In this particulate matter a close mechanical contact between the substance(-s) has been obtained. An example of equipment is Minipactor® or a Gerteis 3W-Polygran from Gerteis Maschinen+Processengineering AG.

Tablet compression according to the present invention takes place without any use of any liquid substances (neither in the form of aqueous solutions, solutions based on organic solutes, or mixtures thereof). In a typical embodiment the resulting core or tablet must have a crushing strength in the range of 10 to 150 N; such as 15 to 125 N, preferably in the range of 20 to 100 N.

A core is thus provided by compression of a powder or a particulate matter. Typically the core has a weight in the range of 75 mg to 2.5 g; such as 80 mg to 1 g; such as 80 mg to 500 mg; such as 100 mg to 300 mg. Preferably, the core is a tablet with a weight in the range of 75 mg to 2.5 g; such as 80 mg to 1 g; such as 80 mg to 500 mg; such as 100 mg to 300 mg. In the present invention, the core is further coated with an enteric coat and optionally a subcoat to obtain the desired tablet formulation.

Tablets according to the present invention may be smaller than conventional tablets, i.e. having a diameter of about 7, preferably about 6 and most preferably about 5 mm or below. In contrast to tablets with a diameter of e.g. 7.5 mm or above, tablets having a smaller diameter will be able to move freely through the pylorus sphincter into the small intestine and thus be less dependent on the gastric emptying.

Subcoating:

Any conventionally used water soluble film forming excipient can be used for subcoating such as a sugar, polyethylene glycol, polyvinylpyrrolidone, polyvinyl alcohol, hydroxypropyl cellulose, methyl cellulose, hydroxymethyl cellulose, hydroxypropyl methylcellulose, polyvinyl acetal diethylenaminoacetate, “Kollicoat IR” (polyvinyl alcohol—polyethylene grycol graft copolymer), etc. Water or any conventionally used organic solvent or a mixture thereof is suitable as a subcoating solvent.

It is a general teaching within the field that the subcoat is critical to the stability of the entire formulation. In contrast to the general teaching, the inventors of the present invention have surprisingly shown that is possible to produce tablets without subcoating having a drug stability compared with tablets with conventional subcoating (table 18 and FIG. 9). As the subcoating process is laborious and time consuming, a production process according to the present invention without laborious subcoating steps is thus far more cost-efficient while still obtaining a product with the desired stability and dissolution properties.

Enteric Coating

Any conventionally used enteric coating polymer can be used such as cellulose acetate phthalate such as Aquacoat® CPD (FMC) or C-A-P NF (Eastman Chemical), polyvinyl acetate phthalate such as Sureteric® (Colorcon), carboxymethylethylcellulose, co-polymerized methacrylic acid/methacrylic acid methyl esters such as Eudragit® L 30 D, or Eudragit® L 12.5 or Eudragit®L 100 (Degussa—Röhm Pharma Polymers) or Kollicoat MAE 30 DP or Kollicoat 100P (BASF) or Acryl-Eze (Colorcon) or Eastacryl 30 D (Eastman Chemical) etc.

Preferred plasticizers include cetanol, triacetin, citric acid esters such as Citroflex® (Pfizer), phthalic acid esters, dibutyl succinate, acetylated monoglyceride, acetyltributyl, acetyltributyl dtrate, acetyltriethyl citrate, benzyl benzoate, calcium stearate, castor oil, cetanol, chlorebutanol, colloidal silica dioxide, dibutyl phthalate, dibutyl sebacate, diethyl oxalate, diethyl malate, diethyl maleate, diethyl malonate, diethyl fumarate, diethyl phthalate, diethyl sebacate, diethyl succinate, dimethylphthalate, dioctyl phthalate, glycerin, glyceroltributyrate, glyceroltriacetate, glyceryl behanate, glyceryl monostearate, hydrogenated vegetable oil, lecithin, leucine, magnesium silicate, magnesium stearate, polyethylene glycol, propylene, glycol, polysorbate, silicone, stearic acid, talc, titanium dioxide, triacetin, tributyl citrate, triethyl citrate, zinc stearate, PEG (polyethylene glycol), etc. Methods for enteric coating are well known in the art such as described in e.g. (Stuart C. Porter in Remmington 21^{st} Ed. 2005, pp 929), hereby incorporated by reference.

Stability

Preferably, at least 95% (w/w) of the declared content of drug substance remains in the tablet formulation according to the present invention after storage at 25° C./60% RH (relative humidity) of a period of 2, 3, 4, or 5 years. Alternatively, the stability can be determined after storage at other conditions according to appropriate ICH guidelines. Methods of assessing stability are described in the Examples.

### BRIEF DESCRIPTION OF DRAWINGS

FIG. 1: Stability test of coated tablets containing Sodium Carbonate, Calcium Carbonate (Sturcal L), or Tri calcium phosphate. Test performed in open petri dishes at 70° and not more than (nmt) 10% relative humidity. Example 6.

FIG. 2: Stability test of coated tablets containing Sodium Carbonate or Calcium Carbonate (Sturcal L) in the mixing ratio's of 1:0.2 or 1:0.8. Test performed in open petri dishes at 70° and not more than (nmt) 10% relative humidity. Example 8.

FIG. 3: Stability test of coated tablets containing Sodium Carbonate (Sturcal L) 1:0.8 based on different sequential order of mixing and roller compaction. Test performed in open petri dishes at 70° and not more than (nmt) 10% relative humidity. Example 10.

FIG. 4: Particle size distribution using two different sieve sizes (1.25 mm and 1.0 mm) during roller compaction. Example 11.

FIG. 5: Impact on dose variation of Roller compactor sieve size illustrated by tablet core dissolution. Example 11.

FIG. 6: Impact on dissolution of amount of subcoat, HPMC E15, applied. Example 12.

FIG. 7: Impact on dissolution of type of subcoat, HPMC E 5 and HPMC E 15. Evaluated on subcoated tablets. Example 12.

FIG. 8: Impact on dissolution of type of subcoat, HPMC E 5 and HPMC E 15. Evaluated for enteric coated tablets. Example 12.

FIG. 9: Stability of batches 13030634 and 31030634 (without the application of a sub coat) at 70° C. in open petri dishes. Example 14.

FIG. 10: Pantoprazole Sodium Sesquihydrate (SEM picture).

FIG. 11: Calcium Carbonate (Sturcal L) (SEM picture).

FIG. 12: Calcum Carbonate (Scoralite) (SEM picture).

FIG. 13: Sodium Carbonate (SEM picture).

FIG. 14: Sodium Carbonate (SEM picture).

FIG. 15: Pantoprazole Sodium Sesquihydrate and Calcium Carbonate (Sturcal L); Mixing followed by slugging (SEM picture).

FIG. 16: Pantoprazole Sodium Sesquihydrate and Calcium Carbonate (Scoralite) Mixing followed by slugging (SEM picture).

FIG. 17: Pantoprazole Sodium Sesquihydrate and Sodium Carbonate; Mixing followed by slugging (SEM picture).

FIG. 18: Pantoprazole Sodium Sesquihydrate and Sodium Carbonate Pre rollercompaction of Pantoprazole, mixing with sodium carbonate, and slugging (SEM picture).

FIG. 19: Impact of disintegrant on tablet core dissolution. Example 18.

It should be noted that, according to the present invention, embodiments and features described in the context of one of the aspects of the present invention also apply to the other aspects of the invention.

The following non-limiting examples are meant to illustrate the present invention.

### EXAMPLES

### Example 1

Dry manufacture of tablets containing pantoprazole or omeprazole. Dry granulation is followed by compressing the resulting particulate matter into tablets.

TABLE 2

| | | Manufactured bathes, no. | | | | | | | | | | | |
|---|---|---|---|---|---|---|---|---|---|---|---|---|---|
| | Raw | | | | | | | | | | | 11 | 12 |
| | Materials* | 1 | 2 | 3 | 4 | 5 | 6 | 7 | 8 | 9 | 10 | a + b | a + b |
| 1 | Pantoprazole | 20.0 | 20.0 | 20.0 | 20.0 | 20.0 | 20.0 | | | | | | |
| | Na 1.5H₂O | | | | | | | | | | | | |
| 1 | Omeprazole | | | | | | | 20.0 | 20.0 | 20.0 | 20.0 | 20.0 | 20.0 |
| | Na | | | | | | | | | | | | |
| 2 | CaCO₃ | 56.4 | 56.4 | | | | | 56.4 | 56.4 | | | | |
| 2 | Na₃PO₄ | | | 56.4 | 56.4 | | | | | 56.4 | 56.4 | | |
| 2 | Na₂CO₃ | | | | | 56.4 | 56.4 | | | | | 56.4 | 56.4 |
| 3 | Sorbitol | 17.6 | | 17.6 | | 17.6 | | 17.6 | | 17.6 | | 17.6 | |
| 3 | Mannitol | | 17.6 | | 17.6 | | 17.6 | | 17.6 | | 17.6 | | 17.6 |
| 4 | MCC | 5.25 | 5.25 | 5.25 | 5.25 | 5.25 | 5.25 | 5.25 | 5.25 | 5.25 | 5.25 | 5.25 | 5.25 |
| 5 | Mg-stearate | 0.75 | 0.75 | 0.75 | 0.75 | 0.75 | 0.75 | 0.75 | 0.75 | 0.75 | 0.75 | 0.75 | 0.75 |

*Amounts in % w/w

TABLE 3
List of ingredients

| | |
|---|---|
| 1 | Pantoprazole sodium sesquihydrate |
| 1 | Omeprazole sodium |
| 2 | CaCO₃ Sturcal L ® |
| 2 | Na₃PO₄ trisodium phosphate |
| 2 | Na₂CO₃ sodium carbonate |
| 3 | Sorbitol |
| 3 | Mannitol |
| 4 | MCC; Cellulose Microcrystalline type 101 ® |
| 5 | Mg-stearate; magnesium stearate |

The drug substance 1) (having a mean particle size of about 7 μm) was mixed by hand with the alkaline substance 2) and with 3).

The resulting mixture of the ingredients 1) to 3) was subjected to roller compaction by use of the following set of parameters:

| | |
|---|---|
| Rpm: | 2.0 |
| Gab size | 2.5 mm |
| Sieve size | 1.25 mm |
| Force | 10 kN/cm |

The resulting particulate matter of the dry granulated ingredients 1) to 3) was admixed with 4) and 5). Thereafter, tablets were compressed of the mixture of ingredients 1) to 5) using a Diaf TM 20 press and 7.5 mm standard concave punch design. Unless otherwise stated, a relatively low compression force was used.

Crushing strength and time of disintegration of the obtained tablets were measured by use of a Schleuniger E2 hardness tester (n=10) and a Sotax DT 2 (n=6). Both tests were performed according to the European Pharmacopoeia.

TABLE 4
Crushing strength and disintegration time

| | Crushing strength | Disintegration time in water |
|---|---|---|
| Batch no. | [N] | [sec.] |
| 1 | 39.5 | 291.0 |
| 2 | 39.5 | 151.0 |
| 3 | 41.3 | 520.0 |
| 4 | 39.9 | 457.8 |
| 5 | 41.1 | 496.5 |
| 6 | 42.1 | 523.0 |
| 7 | 32.4 | 281.8 |
| 8 | 30.9 | 477.5 |
| 9 | 36.0 | 300 |
| 10 | 38.0 | 284.7 |
| 11a | 45.3 | 269.7 |
| Low Comp. | | |
| Force | | |
| 11b | 90.6 | 301.8 |
| High Comp. | | |
| Force | | |
| 12a | 40.7 | 256.8 |
| Low Comp. | | |
| Force | | |
| 12b | 61.7 | 271.2 |
| High Comp. | | |
| Force | | |

The results from table 4 show that tablets containing pantoprazole or omeprazole having a satisfactory crushing strength and disintegration time can be manufactured from a dry manufacturing process based on a particulate matter provided by dry granulation resulting from roller compaction.

Furthermore, batches 11a+b and 12a+b illustrate that the crushing strength can be increased without any significant influence on the disintegration time. This means that the tablets are of a quality that allows the application of a standard enteric coating using standard coating equipment and parameters. Optionally a sub coat comprising a standard water-soluble film like HPMC (Hydroxypropyl methylcellulose) can be used to protect the enteric coat from the alkaline reacting core.

### Example 2

Crushing strength and disintegration time resulting from compression of particulate matter based on roller compaction

TABLE 5

Manufactured
batches, no.

| | Raw Materials | 13 | 14 |
|---|---|---|---|
| 1 | Pantoprazole sodium | 21.3 | 21.3 |
| | sesquihydrate | | |
| | (Mean particle size around 7 μm) | | |
| 2 | Na₃PO₄ trisodium phosphate | 60.0 | 60.0 |
| 3 | Sorbitol | 18.7 | — |
| 3 | Mannitol | — | 18.7 |

*: Amounts in % w/w

1) was mixed by hand with 2) and 3).

The resulting mixture of the ingredients 1) to 3) was roller compacted by use of the following set of parameters:

| | |
|---|---|
| Rpm: | 2.0 |
| Gab size | 2.5 mm |
| Sieve size | 1.25 mm |
| Force | 10 kN/cm |

The particulate matter resulting from roller compaction of the ingredients 1) to 3) was compressed into tablets using a Diaf TM 20 press and 7.5 mm standard concave punch design. Crushing strength and time of disintegration of the obtained tablets were measured by use of a Schleuniger E2 hardness tester (n=10) and a Sotax DT 2 disintegrations tester (n=6).

TABLE 6
Crushing strength and disintegration time

| Batch no. | Crushing strength [N] | Disintegration time [sec.] |
|---|---|---|
| 13 | 42.6 | 443.0 |
| 14 | 52.6 | 486.7 |

The results from table 6 show that tablets containing pantoprazole having a satisfactory crushing strength and disintegration time can be manufactured by compression of a particulate matter provided by a dry granulation process based on roller compacted granulates, without the addition of further excipients (apart from a dry binder). Addition of magnesium stearate could, however, be advantageous in production scale.

The tablets are thus of a quality that allows the application of a standard enteric coating using standard coating equipment and parameters. Optionally a sub coat comprising a standard water-soluble film like HPMC can be used to protect the enteric coat from the alkaline reacting core.

### Example 3

Crushing strength and disintegration time of tablets resulting from direct compression of mixtures of pantoprazole and compactable alkaline excipient

TABLE 7

| | | D(v; 0.5) | | |
|---|---|---|---|---|
| | Raw material | [μm] | 10060531 | 10060532 |
| 1 | Pantoprazole sodium | 7 | 20.00 | 20.00 |
| | sesquihydrate | | | |
| 2 | Trisodium phosphate, | 203 | 79.26 | 71.34 |
| | coarse | | | |
| 2 | Trisodium phosphate, | 40 | | 7.92 |
| | fine | | | |
| 3 | Magnesium stearate | | 0.74 | 0.74 |

*: Amounts in % w/w

The drug substance 1) and the ingredient 2) were mixed by hand followed by admixing of 3). The mixture of ingredients 1) to 3) was compressed into tablets using a Diaf TM 20 press and 7.5 mm standard concave punch design.

Crushing strength and time of disintegration of the obtained tablets were measured by use of a Schleuniger E2 hardness tester (n=10) and a Sotax DT 2 disintegration tester (n=6).

TABLE 8
Crushing strength and disintegration time

| | | Disintegration time | Mass variation |
|---|---|---|---|
| Batch no. | Crushing strength [N] | [sec.] | [s.rel - %] |
| 10060531 | 37.4 | 657 | 0.96 |
| 10060532 | 41.2 | 631 | |

The results shown in table 8 show that tablets containing pantoprazole having a satisfactory crushing strength and disintegration time result from direct compression of the compactable alkaline substance. The mass variation illustrates that the flowability of the mixture of ingredients 1) to 3) is acceptable. It is conceivable that the coarse alkaline substance functions both as a filler substance and an alkaline substance in this formulation.

The resulting tablets are of a quality that allows the application of a standard enteric coating using standard coating equipment and parameters. Optionally a sub coat comprising a standard water soluble film like HPMC can be used to protect the enteric coat from the alkaline reacting core.

### Example 4

Manufacture of tablets having a diameter of 5 mm based on a particulate matter resulting from dry granulation in a roller compactor

TABLE 9

| | Raw | | | | | | | | |
|---|---|---|---|---|---|---|---|---|---|
| | Mate- | | | | | | | | |
| | rials* | 1 | 2 | 3 | 4 | 5 | 6 | 7 | 8 |
| 1 | Panto- | 40 | 40 | 40 | 40 | | | | |
| | prazole | | | | | | | | |
| | sodium | | | | | | | | |
| | 1.5H₂O | | | | | | | | |
| 1 | Ome- | | | | | 40 | 40 | 40 | 40 |
| | prazole | | | | | | | | |
| | sodium | | | | | | | | |
| 2 | Na₃PO₄ | | 40 | 40 | | 40 | 40 | | |
| 2 | Na₂CO₃ | 40 | | | 40 | | | 40 | 40 |
| 3 | Sorbitol | | | 18.25 | 18.25 | 18.25 | | 18.25 | |
| 3 | Mannitol | 18.25 | 18.25 | | | | 18.25 | | 18.25 |
| 4 | Mg- | 0.75 | 0.75 | 0.75 | 0.75 | 0.75 | 0.75 | 0.75 | 0.75 |
| | stearate | | | | | | | | |

Amounts in % w/w

TABLE 10
List of ingredients

| | |
|---|---|
| 1 | Pantoprazole sodium sesquihydrate |
| 1 | Omeprazole sodium |
| 2 | Na₃PO₄ trisodium phosphate |
| 2 | Na₂CO₃ sodium carbonate |
| 3 | Sorbitol |
| 3 | Mannitol |
| 4 | Mg-stearate; magnesium stearate |

The drug substance 1) was mixed by hand with 2) and 3).

The resulting mixture of the ingredients 1) to 3) was dry granulated by roller compaction by use of the following set of parameters:

| | | |
|---|---|---|
| Rpm: | 2.0 | |
| Gab size | 2.5 | mm |
| Sieve size | 1.25 | mm |
| Force | 10 | kN/cm |

The resulting particulate matter of the dry granulated ingredients 1) to 3) was admixed with 4). The resulting mixture of the ingredients 1) to 4) was compressed into tablets using a 5.0 mm standard concave punch design. The resulting tablets are of a quality that allows applying a standard enteric coat using standard coating equipment and parameters. Optionally a sub coat consisting of a standard water soluble film like HPMC can be used to protect the enteric coat from the alkaline reacting core.

### Example 5

Dry manufacture of tablets containing pantoprazole and alkaline excipients followed by application of a sub coat and an enteric coating.

TABLE 11
Composition of tablet core % w/w:

| | 07030631 | 10030631 | 13030634 | 13030636 |
|---|---|---|---|---|
| Batch no. | “33C” | “34C” | “35C” | “36C” |
| Granulate by roller | | | | |
| compaction | | | | |
| Pantoprazole, 1½ H₂O | 13.3 | 11.7 | 14.1 | 12.9 |
| Sodium carbonate | 13.3 | | 55.2 | — |
| Calcium carbonate | — | 54.9 | — | — |
| (Sturcal L) | | | | |
| TriCalciumPhosphate | | | | 60.6 |
| Pantoprazole:alkaline | 1:1 | 1:4.7 | 1:3.9 | 1:4.7 |
| excipient | | | | |
| Mannitol | — | 13.5 | — | 14.9 |
| Crospovidone | 0.4 | 1.1 | 1.1 | 1.2 |
| Tablet core excipients | | | | |
| DiCafos A | 62.7 | 8.5 | 19.2 | — |
| Crospovidone | 9.7 | 9.7 | 10.0 | 9.8 |
| Mg Stearate | 0.5 | 0.4 | 0.5 | 0.5 |

TABLE 12
Composition of sub coat [% w/w]:

| | Coating liquid | Dry film |
|---|---|---|
| Raw materials | composition | composition |
| Hypromellose 15 | 5 | 55.6% |
| Propylenglycol | 1 | 11.1% |
| Talc | 3 | 33.3% |
| Water, purified | 91 | |
| % Plasticizer of polymer | | 20.0% |

TABLE 13
Composition of enteric coat [% w/w]::

| | Coating liquid | Dry film |
|---|---|---|
| Raw materials | composition | composition |
| Methacryl acid-acryl | 41.76 | 62.49 |
| Copolymer disp. 30% | | |
| Triethylcitrate | 1.25 | 6.24 |
| Talc | 6.27 | 31.28 |
| Water, purified | 50.92 | |
| % Plasticizer of Polymer | | 10.0 |

TABLE 17
Approximately amount of film dry matter applied [% w/w]:

| | 07030631 | 10030631 | 13030634 | 13030636 |
|---|---|---|---|---|
| Coat | “33C” | “34C” | “35C” | “36C” |
| Theoretical applied | 9.7 | 10.3 | 10.9 | 9.0 |
| amount of sub coat | | | | |
| [mg polymer/cm^{2]} | | | | |
| Theoretical applied | 5.5 | 5.4 | 5.3 + 2 | 5.7 |
| amount of enteric coat | | | | |
| [mg polymer/cm^{2]} | | | | |

Pantoprazole was mixed with the alkaline excipient in a tumble mixer together with Crosspovidone and mannitol followed by roller compaction as described in example 1. The remaining tablet core excipients were admixed and tablets were compressed by use of a Korsch PH106 tablet press and 6 mm concave punches aiming at a mean weight of 160 mg and a crushing strength of 50 N. Thereafter the tablet cores were coated with the sub coat followed by the enteric coat by use of a lab-scale Combi Coata. The obtained coated tablets were used for stability testing as described in example 6.

### Example 6

(Stability Testing)

A stability testing program with batches obtained in example 5 was performed. The batches were stored at accelerated stability testing conditions (open petri dishes at 70° C. and not more than 10% relative humidity for three months). Such conditions probably correspond to shelf life stability testing of at least two years.

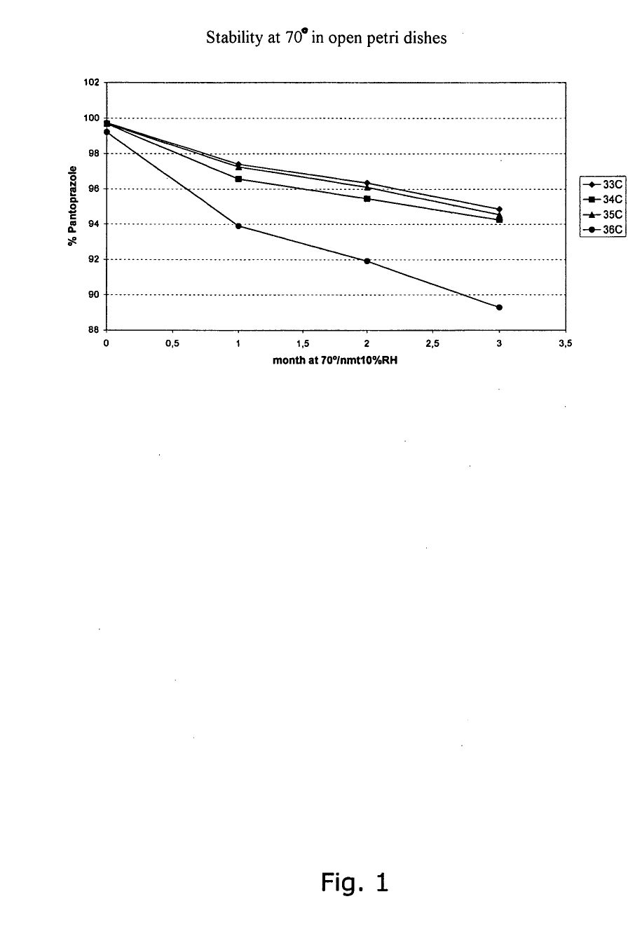

The analytical method is as follows: 10 tablets are transferred to a 200 ml volumetric flask. 150 ml mobile phase (the initial composition) is added and sample is shaken for 90 minutes. After the solutions pH-values have been adjusted to 8.0, mobile phase is added to the mark. The sample solution is filtered through 0.45 μm filter and analysed by reverse phase HPLC in order to quantify the amount of Pantoprazole as well as degradation products thereof. The amount is given in % of total area, see table 19. Furthermore, in FIG. 1 is shown the amount of pantoprazole as a function of time.

TABLE 18
HPLC method parameters

| Mobile | ammonium phosphate buffer: methanol:acetonitril | | | |
|---|---|---|---|---|
| phase | | | | |
| | Time | % Buffer | % Methanol | % Acetonitril |
| Gradient profile | 0 min | 65 | 10 | 25 |
| | 25 min | 30 | 10 | 60 |
| | 30 min | 30 | 10 | 60 |
| | 31 min | 65 | 10 | 25 |

| Flow | 1.0 ml/min | |
|---|---|---|
| Column | Waters Spherisorb, 250 × 4.6 mm, 5 μm particle | |
| | size | |
| Column temperature | 30° C. | |
| Auto sampler | 4° C. | |
| temperature | | |
| Detection | UV-290 nm | |
| Injection volume | 10 μl | |
| Run time | 40 min. | |
| Approx. Retention | Pantoprazole | 10.7 min |
| time | Impurity B | 6.6 min |
| | Impurity A | 13.4 min |

TABLE 19
Results of stability study

| | | 07030631 | 10030631 | 13030634 | 13030636 |
|---|---|---|---|---|---|
| Batch | | “33C” | “34C” | “35C” | “36C” |
| Pantoprazole:sodium carbonate | | 1:1 | | 1:3.9 | |
| Pantoprazole:calcium carbonate | | | 1:4.7 | | |
| Pantoprazole:calcium phosphate | | | | | 1:4.7 |
| Pantoprazole:DiCafos A | | 1:4.7 | 1:0.7 | 1:1.4 | |
| Pantoprazole in | 25/60 C 1 month | 99.7 | 99.7 | 99.7 | 99.2 |
| % area | 70/10 OP 1 month | 97.4 | 96.6 | 97.3 | 93.9 |
| | 70/10 OP 2 months | 96.4 | 95.5 | 96.1 | 91.9 |
| | 70/10 OP 3 months | 94.9 | 94.3 | 94.6 | 89.3 |
| | 40/75 3 months | 99.4 | 99.4 | 99.5 | 97.2 |
| Degradation products | 25/60 C 1 month | 0.18 | 0.15 | 0.20 | 0.56 |
| in % area | 70/10 OP 1 month | 2.2 | 3.2 | 2.3 | 6.0 |
| | 70/10 OP 2 months | 3.3 | 4.0 | 3.6 | 7.6 |
| | 70/10 OP 3 months | 4.3 | 4.8 | 4.4 | 9.3 |
| | 40/75 3 months | 0.5 | 0.5 | 0.5 | 2.6 |

70/10 OP: 70° C./10% RH in Open petri dishes
25/60: 25° C./60% RH in closed containers
40/75: 40° C./75% RH in closed containers

According to table 19 and FIG. 1, use of Ca₅(PO₄)₃OH as alkaline substance result in a relatively larger degree of pantoprazole degradation compared with use of Na₂CO₃, CaCO₃, and DiCafos A as alkaline excipients. The stability data for the stressed stability testing conditions (70° C. open Petri dishes, 70/10 OP) probably corresponds to a shelf life of at least two years.

### Example 7

Dry manufacture of tablets containing pantoprazole and Sodium carbonate or calcium carbonate and a disintegrant followed by application of a sub coat and an enteric coating.

TABLE 20
Composition of tablet core % w/w:

| | | 05050632 | 29050636 | 29050640 |
|---|---|---|---|---|
| | 01050633 3F-C | 2F-C | 7F-C | 8F-C |
| Granulate by roller | | | | |
| compaction | | | | |
| Pantoprazole, | 14.1 | 14.1 | 14.1 | 14.1 |
| 1½ H₂O | | | | |
| Sodium carbonate | 2.82 | 11.3 | | |
| Calcium carbonate | | | 2.82 | 11.3 |
| (Sturcal L) | | | | |
| Pantoprazole: | 1:0.2 | 1:0.8 | 1:0.2 | 1:0.8 |
| alkaline excipient | | | | |
| Tablet core | | | | |
| excipients | | | | |
| DiCafos A | 69.6 | 61.1 | 69.6 | 61.1 |
| Crosspovidone | 13.0 | 13 | 13.0 | 13 |
| Mg Stearate | 0.5 | 0.5 | 0.5 | 0.5 |

Based on the tablet core compositions listed above coated tablets were manufactured as described in example 5 with the following exception:

The pantoprazole was pre-rollercompacted prior to mixing with the alkaline excipient by use of the following parameters
Rpm: 2.0
Gap size: 1.0 mm
Sieve size: 1.25 mm
Force: 4 kN/cm

The pre-roller compaction leads to formation of pantoprazole granules.

The obtained coated tablets were used for stability testing with the purpose of investigating the impact of lower amounts of alkaline material than used in example 5 and the use of pre-rollercompaction of the pantoprazole. The stability testing is disclosed in example 8.

### Example 8

Stability Testing

A stability program, including batches obtained in example 7 was performed. The batches were stored in open petri dishes at 70° C. and not more than 10% RH for up to six weeks.

The analytical method is as described in example 6

TABLE 21
Result of stability study

| | | 01050633 | 05050632 | 29050636 | 29050640 |
|---|---|---|---|---|---|
| batch | | 3F-C | 2F-C | 7F-C | 8F-C |
| Pantoprazole:sodium carbonate | | 1:0.8 | 1:0.2 | | |
| Pantoprazole:calcium carbonate | | | | 1:0.2 | 1:0.8 |
| Pantoprazole:DiCafos A | | 1.5 | 1:4.3 | 1:5 | 1:4.3 |
| Pantoprazole | 25/60 C 2 weeks | 99.7 | 99.8 | 99.8 | 99.1 |
| in % area | 70/10 OP 2 weeks | 98.3 | 97.9 | 98.3 | 98.1 |
| | 70/10 OP 6 weeks | 97.5 | 97.2 | | |
| Degradation products | 25/60 C 2 weeks | 0.20 | 0.15 | 0.10 | 0.52 |
| in % area | 70/10 OP 2 weeks | 1.2 | 1.6 | 1.4 | 1.2 |
| | 70/10 OP 6 weeks | 2.0 | 2.4 | | |

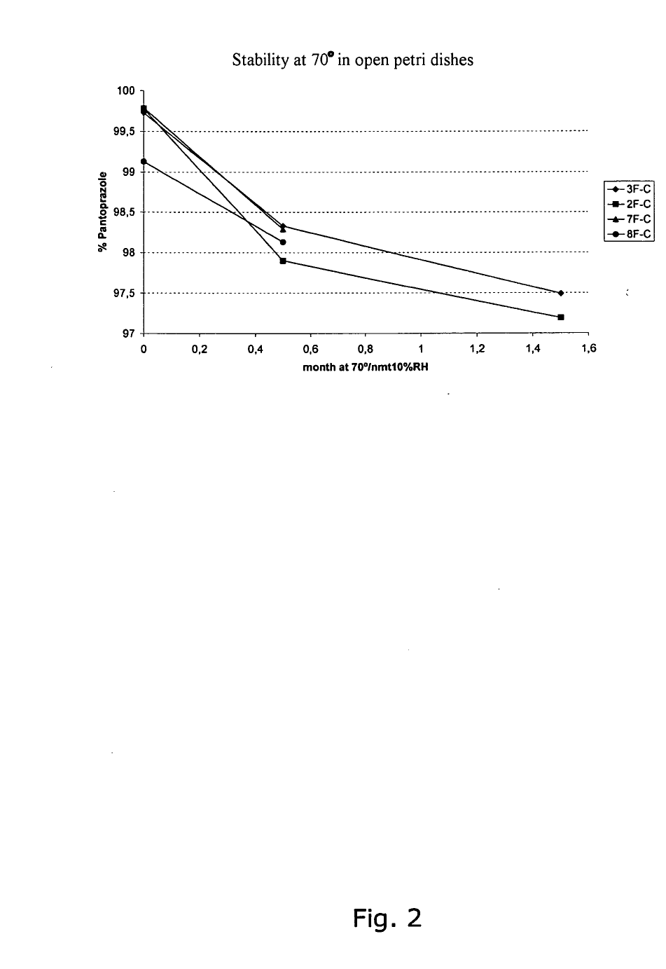

It appears in table 21 and FIG. 2 that use of sodium carbonate and calcium carbonate as alkaline excipient at the mixing ratios of 1:0.2 and 1:0.8 result in nearly identical and acceptable degradation ratios when tested at 70° C. and not more than 10% RH for up to six weeks. It is surprising that the solubility of the alkaline material apparently does not affect stability of the benzimidazole composition (calcium carbonate is poorly soluble in water).

### Example 9

Dry manufacture of tablets containing pantoprazole and alkaline excipients focusing on order of mixing and roller compaction followed by application of a sub coat and an enteric coating.

TABLE 22
Composition of tablet core % w/w:

| Granulate by roller compaction | |
|---|---|
| Pantoprazole, 1½ H₂O | 14.1 |
| Sodium carbonate | 11.3 |
| Pantoprazole:alkaline excipient | 1:0.8 |
| Tablet core excipients | |
| DiCafos A | 61.1 |
| Crospovidone | 13 |
| Mg Stearate | 0.5 |

The composition shown in table 22 was mixed and roller compacted in different sequential order as shown in table 23

TABLE 23
Sequential order of mixing and roller compaction:

| | 1 | 2 | 3 | |
|---|---|---|---|---|
| | Mixing | Pre-roller | Mixing | |
| Batch no. | with | compaction | with | 4 |
| (Of coated | alkaline | (solely | alkaline | Roller |
| tablets) | excipient | pantoprazole) | excipient | compaction |
| 01050633 3F-C | Not used | X | X | X |
| 05050635 | X | Not used | Not used | X |
| 05050638 12F-C | Not used | X | X | Not used |

Mixing and roller compaction were carried out as described in example 5.

The remaining tablet core excipients (Dicafos A, Crosspovidone and Mg-stearate) were admixed and tablets were compressed and coated in accordance with example 5.

The batches 01050633 and 05050638 were used for stability testing in example 10 and 05050635 was used for comparison with batches of example 16 with the purpose of evaluating excipient homogeneity of granules.

### Example 10

Stability Testing

A stability program, including batches mentioned in example 9 was performed. The batches were stored in open petri dishes at 70° C. for 2 weeks.

The analytical method is as described in example 6

TABLE 24
Results of stability testing:

| | | batch | |
|---|---|---|---|
| | | 01050633 3F-C | 05050638 12F-C |
| Pantoprazole:sodium carbonate | | 1:0.8 | 1:0.8 |
| Pantoprazole:DiCafos A | | 1:5 | 1:4.3 |
| | | Pre- | |
| | | rollercompacting, | Pre- |
| | | mixing, | rollercompacting, |
| | | rollercompacting | mixing |
| Pantoprazole | 25/60 C 2 weeks | 99.7 | 99.5 |
| in % area | 70/10 OP 2 weeks | 98.3 | 97.2 |
| | 70/10 OP 6weeks | 97.5 | |
| Degradation | 25/60 C 2 weeks | 0.20 | 0.33 |
| products in | 70/10 OP 2 weeks | 1.2 | 2.9 |
| % area | 70/10 OP 6 weeks | 2.0 | |

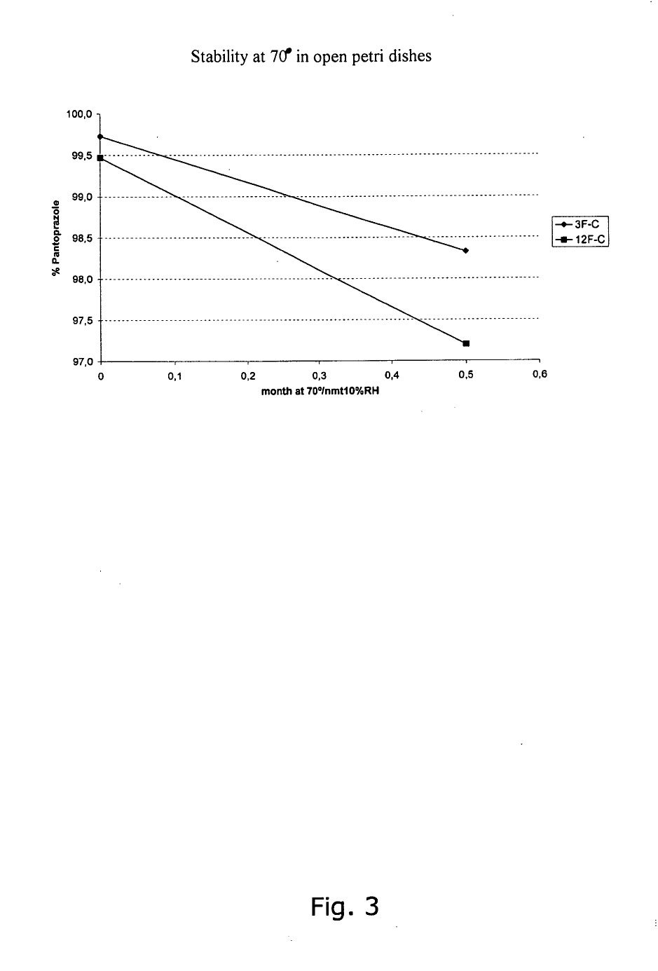

It appears in table 24 and FIG. 3 that panoprazole degradation is relatively small when pantoprazole is pre-roller compacted, mixed with the alkaline excipient and then roller compacted again. The pantoprazole degradation is relatively high when pantoprazole is pre-roller compacted and mixed with the alkaline excipient without including a step of roller compacting pantoprazole and alkaline substance together. The impact on stability is seen already after two weeks.

### Example 11

Dry manufacture of tablets containing pantoprazole and alkaline excipients. Roller compaction has been carried out using two different sieve sizes. Tableting was followed by application of a sub coat and an enteric coating.

TABLE 25
Composition of tablet core % w/w:

| Granulate by roller compaction | |
|---|---|
| Pantoprazole, 1½ H₂O | 14.1 |
| Sodium carbonate | 2.82 |
| Pantoprazole:alkaline excipient | 1:0.2 |
| Tablet core excipients | |
| DiCafos A | 69.6 |
| Crospovidone | 13.0 |
| Mg Stearate | 0.5 |

Based on the tablet core compositions listed above coated tablets were manufactured as described in example 5 with the following exception:

The pantoprazole was pre-rollercompacted prior to mixing with the alkaline excipient by use of the following parameters
Rpm: 2.0
Gap size: 1.0 mm
Sieve size: 1.25 mm (used for batch 29050641) or 1.0 mm (used for batch 29050645)
Force: 4 kN/cm

The pre-roller compacted pantoprazole and the sodium carbonate were mixed by use of a tumble-mixer and roller compacted using the following parameters:
Rpm: 2.0
Gap size: 2.5 mm
Sieve size: 1.25 mm (used for batch 29050641) or 1.0 mm (used for batch 29050645)
Force: 10 kN/cm

The remaining tablet core excipients were admixed and tablets were compressed and coated in accordance with example 5.

Evaluation of the impact of the sieve size was based on sieve analysis and dissolution testing. Dissolution was carried out by use of the following method:

TABLE 26
Dissolution method.
USP/Ph.Eur Dissolution apparatus 1

| | |
|---|---|
| Spindle | Basket |
| Rotation | 150 rpm |
| Temperature | 37° C. ± 0.5° C. |
| Filter | Whatman GF/F (0.7 μm) |
| Dissolution medium 0-120 minutes | 600 ml 0.1 N HCl |
| After 120 minutes the dissolution | 200 ml 0.20 M Na₃PO₄ added to the |
| medium is changed to pH 6.8 | vessel (method A, USP) |
| Detection, UV | 288 nm |
| Sampling time | The absorbance is measured by each |
| | 10 minutes at 288 nm |

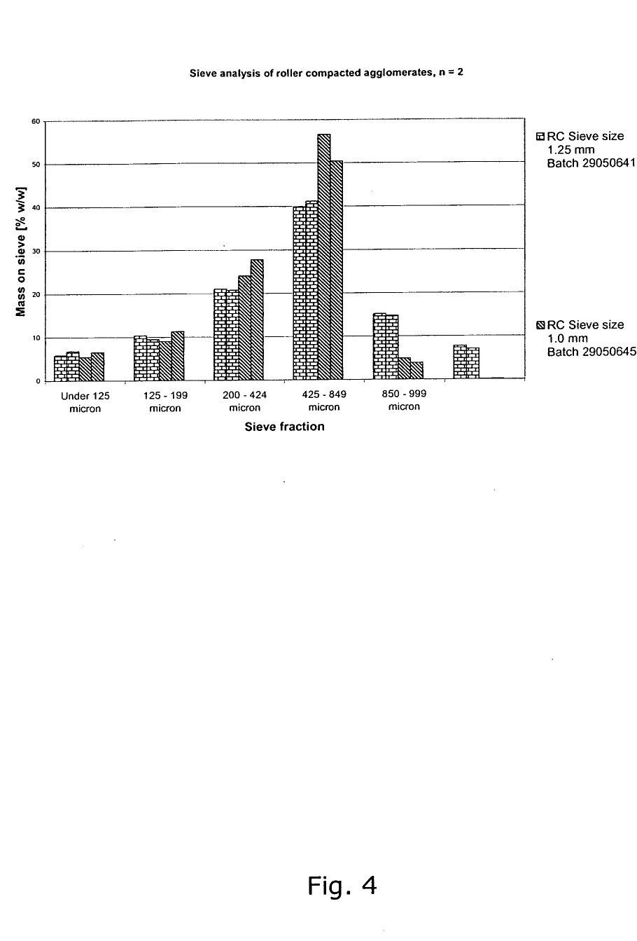

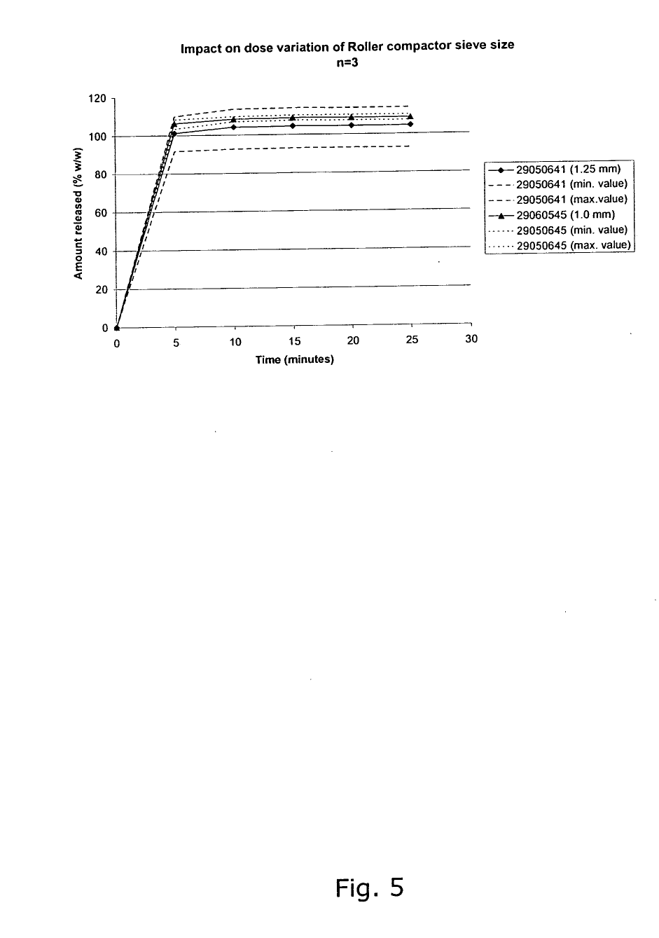

From FIG. 4 it can be seen that the use of a sieve size of 1.0 mm results in a relatively narrow particle size distribution compared to sieve size 1.25. The narrow size distribution results in a better dose variation of pantoprazole as can be seen form FIG. 5.

### Example 12

Dry manufacture of tablets containing pantoprazole and alkaline excipients followed by application of different types and amounts of sub coat.

TABLE 27
Composition of tablet core % w/w:

| Granulate by roller compaction | |
|---|---|
| Pantoprazole, 1½ H₂O | 14.1 |
| Sodium carbonate | 55.2 |
| Pantoprazole:alkaline excipient | 1:3.9 |
| Crospovidone | 1.1 |
| Tablet core excipients | |
| DiCafos A | 19.2 |
| Crospovidone | 10.0 |
| Mg Stearate | 0.5 |

Tablet cores according to the above mentioned composition were manufactured as described in example 5. Sub coat was applied as laid out in example 5 with the exception of variation in applied amount and type of polymer as shown in table 28:

TABLE 28
Amount (approximately) and type of sub coat applied:

| | | Theoretical amount of HPMC |
|---|---|---|
| Batch no. | HPMC type | [mg/cm²] |
| 13030634 | E15 | 10.9 (1/1 amount) |
| 31030633 (C) | E15 | 5.3 (½ amount) |
| 21040634 | E5 | 9.9 (1/1 amount) |
| 31030631 | — | — |

Enteric coat was applied as described in example 5.

Evaluation of the impact of amount and type of sub coat was based following dissolution results obtained obtained in example 11. However, tablets only coated with a sub coat were analysed solely in a dissolution medium with pH 6.8.

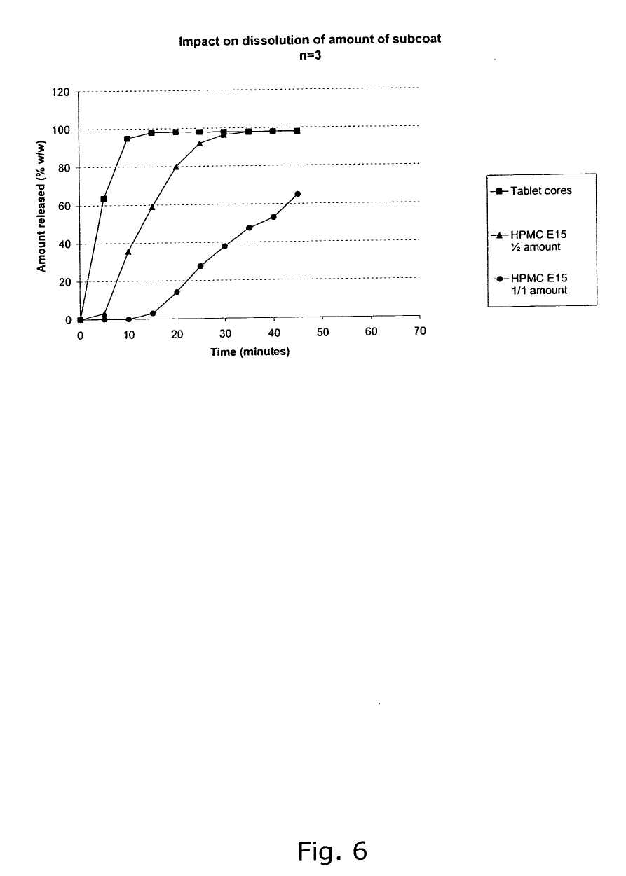

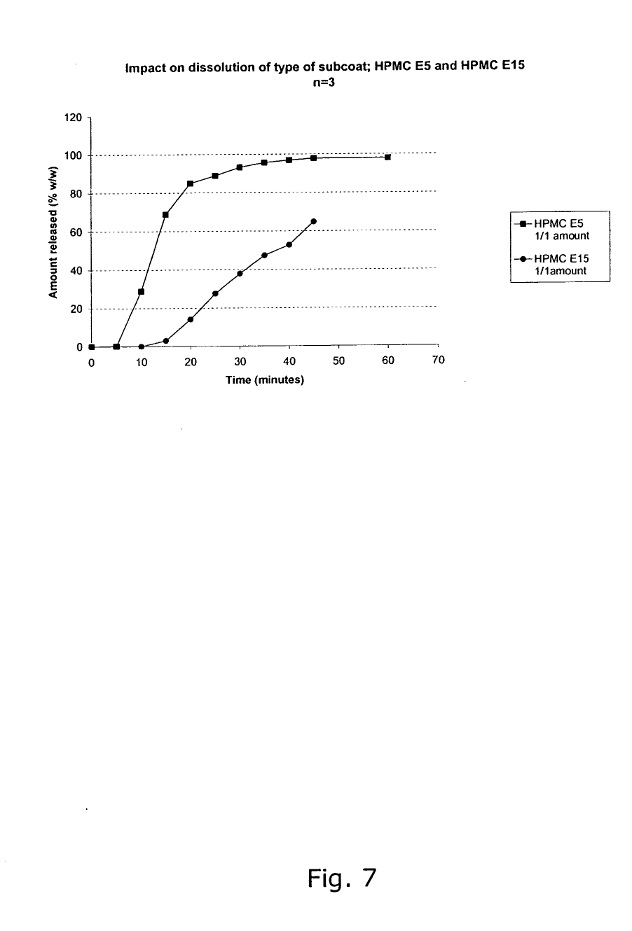

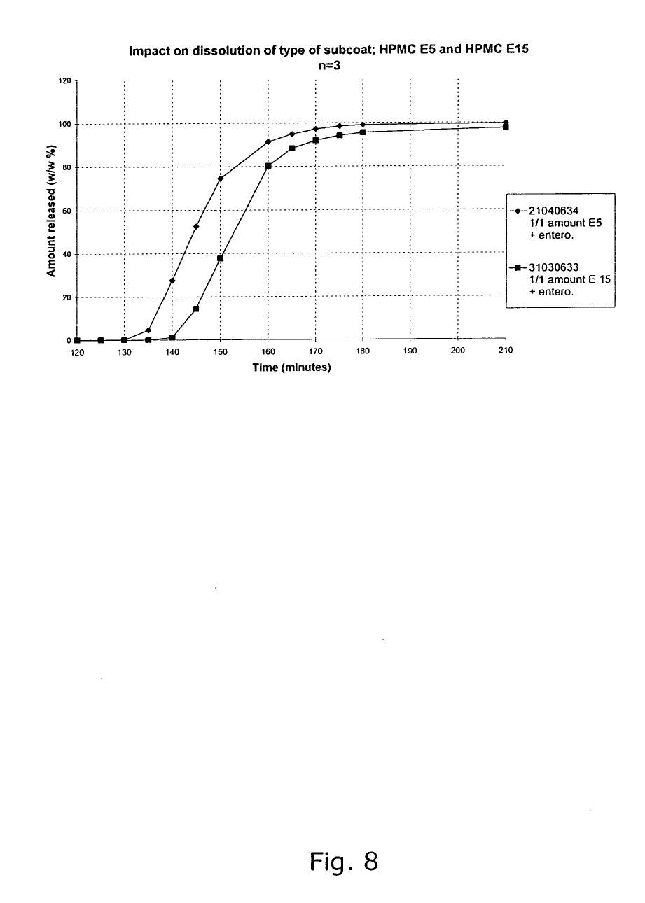

FIGS. 6, 7 and 8 disclose the impact of type of sub coat on the dissolution rate form both sub coated tablets and sub+enteric coated tablets. A HPMC E5 based sub coat results in a relatively quick dissolution rate.

### Example 13

Dry manufacture of tablets containing pantoprazole and alkaline excipients followed by application of an enteric coating.

Tablet cores of batch 13030634 and 31030634 were manufactured as described in example 12. The tablet cores of batch 13030634 were enteric coated as described in example 5. Batch 31030634 was produced with an enteric coating but without the application of a sub coat. The enteric coated tablets were used for stability testing in example 14.

### Example 14

Stability Testing

A stability program, including batches mentioned in example 13 was performed. The batches were stored in open petri dishes at 70° C. for respectively 2 and 3 months.

The analytical method is as described in example 6

TABLE 29
Results of stability testing

| | | | 31030634 |
|---|---|---|---|
| Batch | | 13030634 | Without sub coat |
| Pantoprazole | 25/60 C | 99.7 | 99.7 |
| in % of area | 1 month | | |
| | 70/10 OP | 97.3 | 97.9 |
| | 1 month | | |
| | 70/10 OP | 96.1 | 97.4 |
| | 2 months | | |
| Degradation products | 25/60 C | 0.20 | 0.22 |
| in % of area | 1 month | | |
| | 70/10 OP | 2.3 | 1.7 |
| | 1 month | | |
| | 70/10 OP | 3.6 | 2.2 |
| | 2 months | | |

70/10 OP: 70° C./10% RH in Open petri dishes
25/60: 25° C./60% RH in closed containers

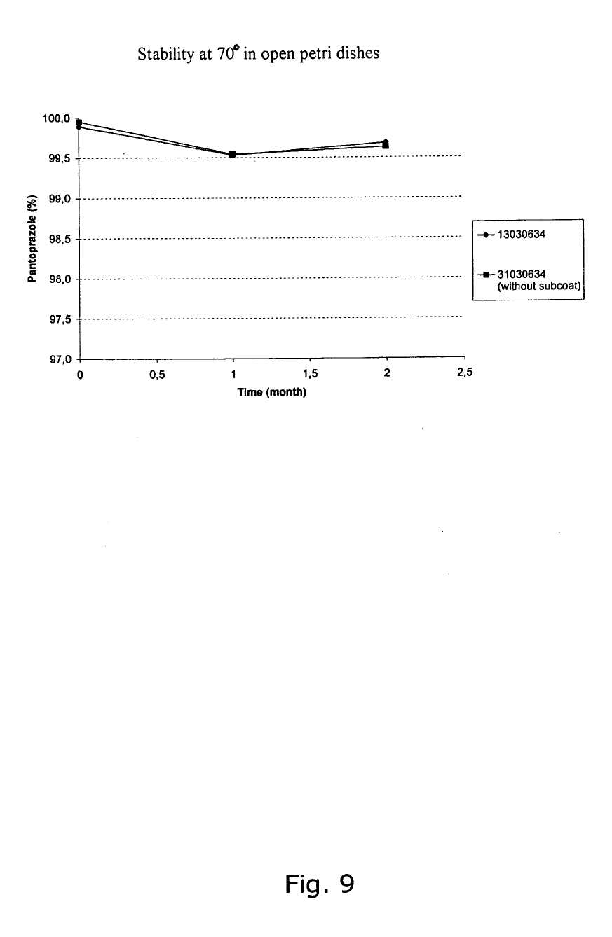

It appears from table 29 and FIG. 9 that the stability (70° C./10% RH in Open petri dishes and 25° C./60% RH in closed containers) of sub-coated tablets is fully comparable with tablets which has not been sub-coated.

### Example 15

Impact of type and amount of alkaline excipient on degradation of pantoprazole in a stress test by the addition of a weakly acidic component (ibuprofen)

TABLE 30
Composition of mixtures [gram]

| Raw materials | Amounts in gram | | | | | | | |
|---|---|---|---|---|---|---|---|---|
| Sodium | 4 | 16 | 20 | 25 | | | | |
| carbonate | | | | | | | | |
| Calcium | | | | | 4 | 16 | 20 | 25 |
| carbonate | | | | | | | | |
| (Sturcal L) | | | | | | | | |
| Ibuprofen | 0.8 | 0.8 | 0.8 | 0.8 | 0.8 | 0.8 | 0.8 | 0.8 |
| Pantoprazole | 20 | 20 | 10 | 5 | 20 | 20 | 10 | 5 |
| 1½ H₂O | | | | | | | | |
| Pantopra- | 1:0.2 | 1:0.8 | 1:2 | 1:5 | 1:0.2 | 1:0.8 | 1:2 | 1:5 |
| zole:alkaline | | | | | | | | |
| excipient | | | | | | | | |

TABLE 31
Composition of mixtures [gram]

| Raw materials | Amounts in gram | | | | | | | |
|---|---|---|---|---|---|---|---|---|
| Calcium | 4 | 16 | 20 | 25 | | | | |
| carbonate | | | | | | | | |
| (Scoralite) | | | | | | | | |
| Tri calcium | | | | | 4 | 16 | 20 | 25 |
| phosphate | | | | | | | | |
| Ibuprofen | 0.8 | 0.8 | 0.8 | 0.8 | 0.8 | 0.8 | 0.8 | 0.8 |
| Pantoprazole | 20 | 20 | 10 | 5 | 20 | 20 | 10 | 5 |
| 1½ H₂O | | | | | | | | |
| Pantopra- | 1:0.2 | 1:0.8 | 1:2 | 1:5 | 1:0.2 | 1:0.8 | 1:2 | 1:5 |
| zole:alkaline | | | | | | | | |
| excipient | | | | | | | | |

Mixtures were made by grinding the raw materials in a steel bowl and subsequently placing the mixture in petri dishes in sealed alu-bags for two weeks at ambient conditions.

The evaluation of this stability test is shown in table 32 (pantoprazole degradation products are coloured). Discoloration is evaluated based on a scale ranging from 1-10 where “1” indicates no discoloration and “10” indicates a severe discoloration.

TABLE 32
Discoloration scale of powder mixtures of table 30 and 31:

| | Mixing | | | |
|---|---|---|---|---|
| | ratio | Mixing ratio | Mixing ratio | Mixing ratio |
| Alkaline material | 1:0.2 | 1:0.8 | 1:2 | 1:5 |
| Sodium carbonate | 9 | 8 | 4 | 1 |
| Calcium carbonate | 10 | 8 | 4 | 1 |
| (Sturcal L) | | | | |
| Calcium carbonate | 10 | 10 | 7 | 7 |
| (Scoralite) | | | | |
| Tri calcium | 9 | 6 | — | 1 |
| phosphate | | | | |

The results in table 32 are discussed in example 16.

### Example 16

Characterisation of alkaline materials.

Alkaline materials:
Calcium carbonate, Sturcal L
Calcium carbonate, Scoralite
Sodium carbonate anhydrate

Alkaline substance raw material particle sizes have been measured by laser light scattering (Malvern) and BET area has been measured by use of a Micromeritics Gemini 2375 at relative target pressures (P/P₀) of 0.1 and 0.2 and 0.3. Samples have been dried for minimum 12 hours at 40° C. prior to the measurements.

TABLE 33
BET-areas

| | BET-area | Particle size |
|---|---|---|
| Raw materials | [m²/g] | D(v 0.5) [μm] |
| Calcium carbonate, Sturcal L | 3.0 | 9 |
| Calcium carbonate, Scoralite | 0.2-0.5 | 29 |
| Sodium carbonate anhydrate | 2.1 | 99 |

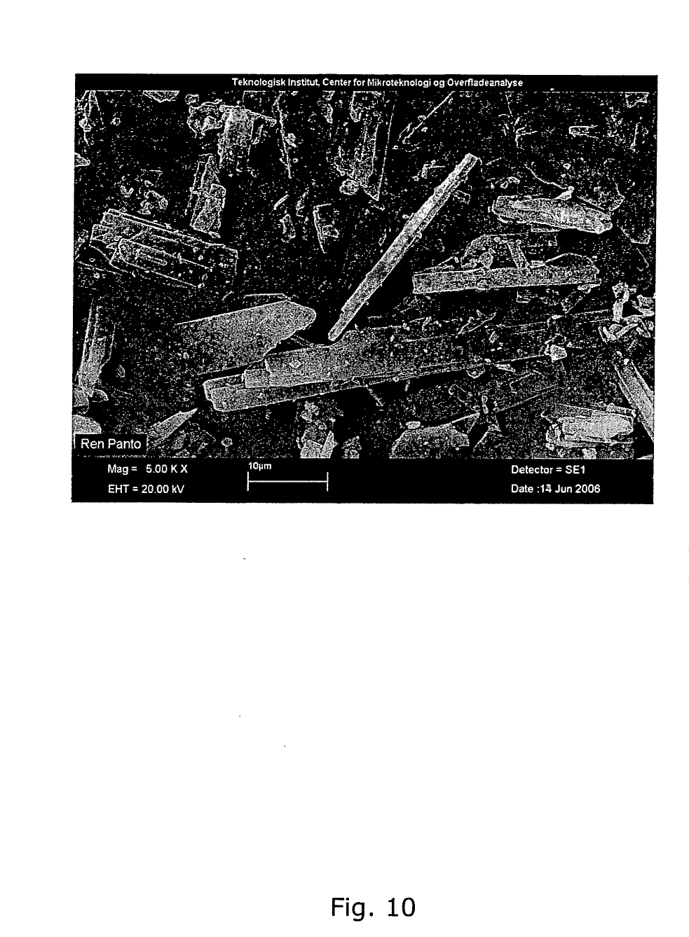

The SEM pictures (FIGS. 10-18) illustrate considerable differences in size and morphology on the alkaline raw materials. It should be noted that even though Sodium carbonate anhydrate has a larger particle size than the Calcium carbonate (Scoralite), sodium carbonate anhydrate has the largest BET area. This difference is further illustrated in example 17 by use of Scanning Electron Microscope pictures. The impact of the BET area and particle size on stability was illustrated in example 15.

The discoloration shown in table 32 demonstrates the impact of BET area (see example 16, table 33) and mixing ratio on pantoprazole stability. A high BET area favours good stability results. It appears from example 16 that a relatively small particle size may not suffice to ensure a satisfactory stability. Relatively big particles can be useful, provided that their porosity leads to a sufficiently high BET area.

Furthermore, it is illustrated that a high amount of alkaline material is preferred. However, the discoloration test cannot reveal smaller amount of degradation product as can be seen by comparison of the addition of tri calcium phosphate in example 6. This means that tri calcium phosphate is not as efficient as e.g. sodium carbonate and calcium carbonate (Sturcal L).

### Example 17

Impact of type and amount of alkaline excipient on homogeneity of a mixture with pantoprazole with alkaline materials.

Composition of powder mixtures
Pantoprazole, 1½ H2O: Calcium carbonate (Sturcal L), 1:0.8
Pantoprazole, 1½ H2O: Calcium carbonate (Scoralite), 1:0.8
Pantoprazole, 1½ H2O (pre-roller compacted): sodium carbonate, 1:0.8

Pantoprazole, 1½ H2O: Calcium carbonate (Sturcal L) and Pantoprazole, 1½ H2O: Calcium carbonate (Scoralite) batches have been mixed in a lab. scale high shear mixer for 1 minute. The Pantoprazole, 1½ H2O (pre-roller compacted) batch was manufactured as described in example 7 prior to mixing with sodium carbonate. The mixing was done as described above.

All the mixtures were slugged on a single punch tableting machine using 11.3 mm flat faced punches. The slugs were evaluated for mixture homogeneity by use of scanning electronic microscopy (SEM). For reference, SEM pictures of the individual raw materials were obtained.

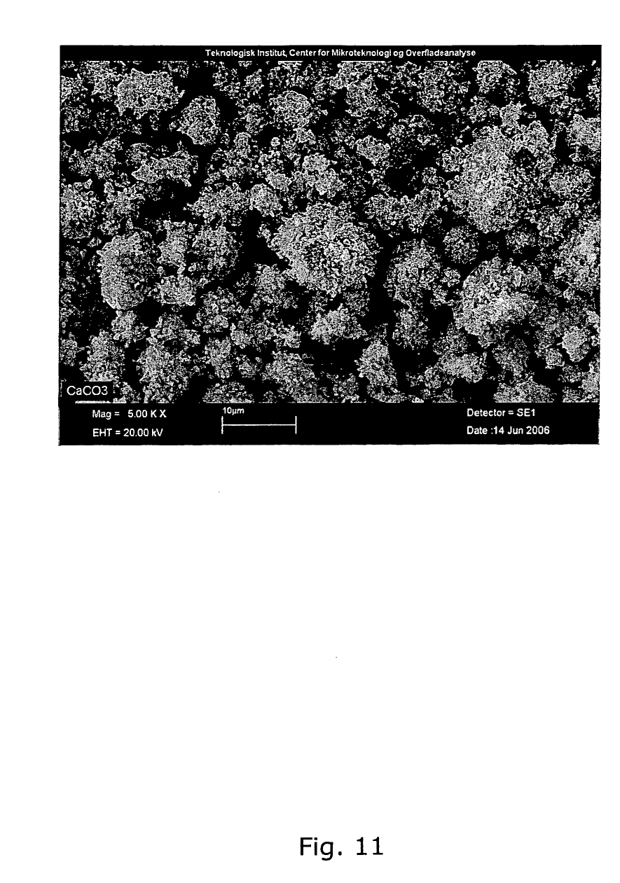

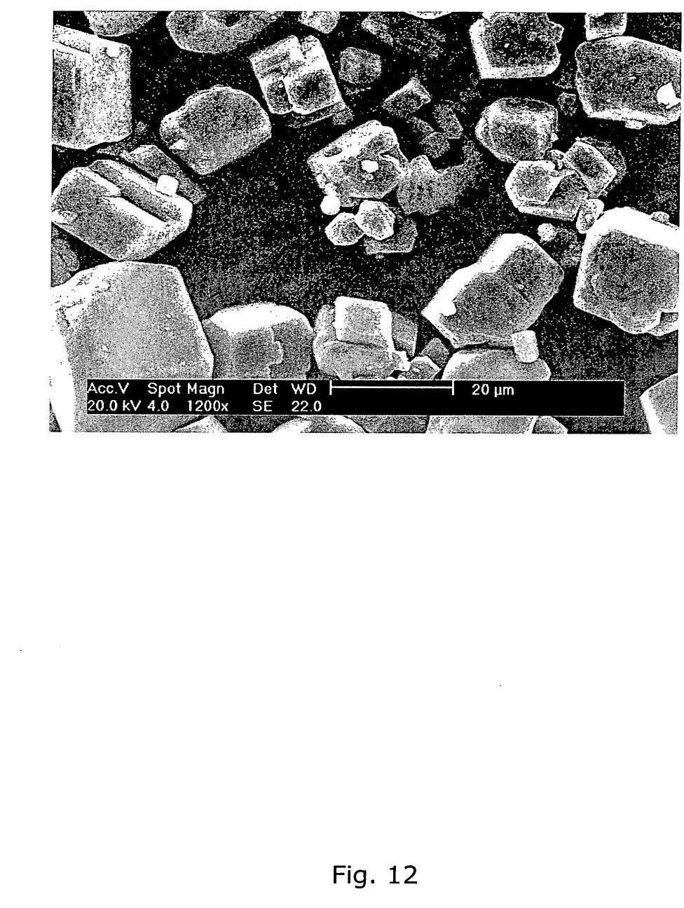

The SEM pictures are shown in the FIGS. 10-18. FIG. 11 and 12 disclose a considerable difference between Calcium carbonate (Sturcal L) and Calcium carbonate (Scoralite). SEM appearances of Sturcal L and Scoralite are in full accordance with the BET areas measured in example 16.

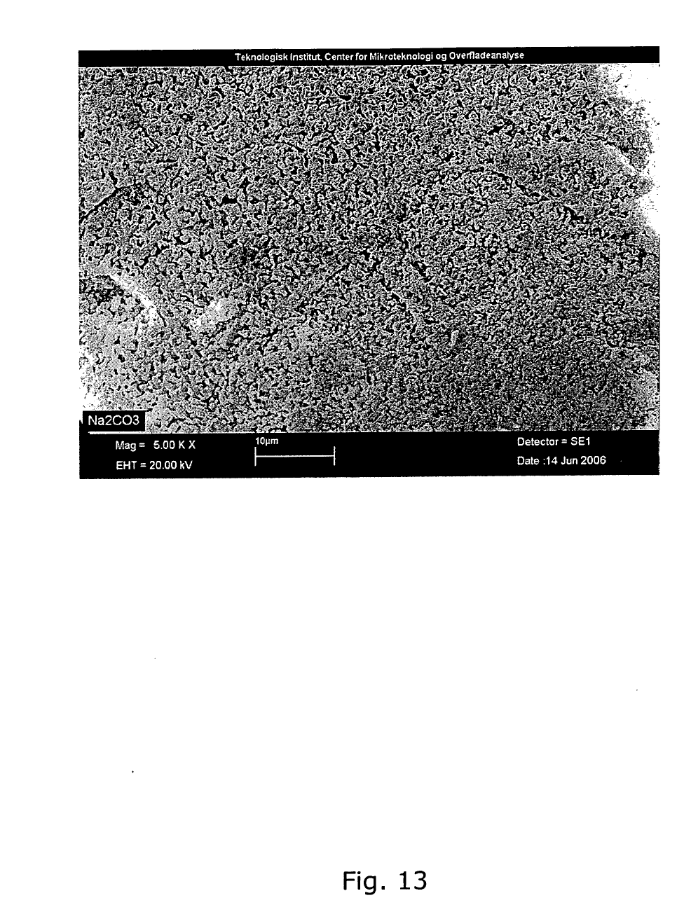

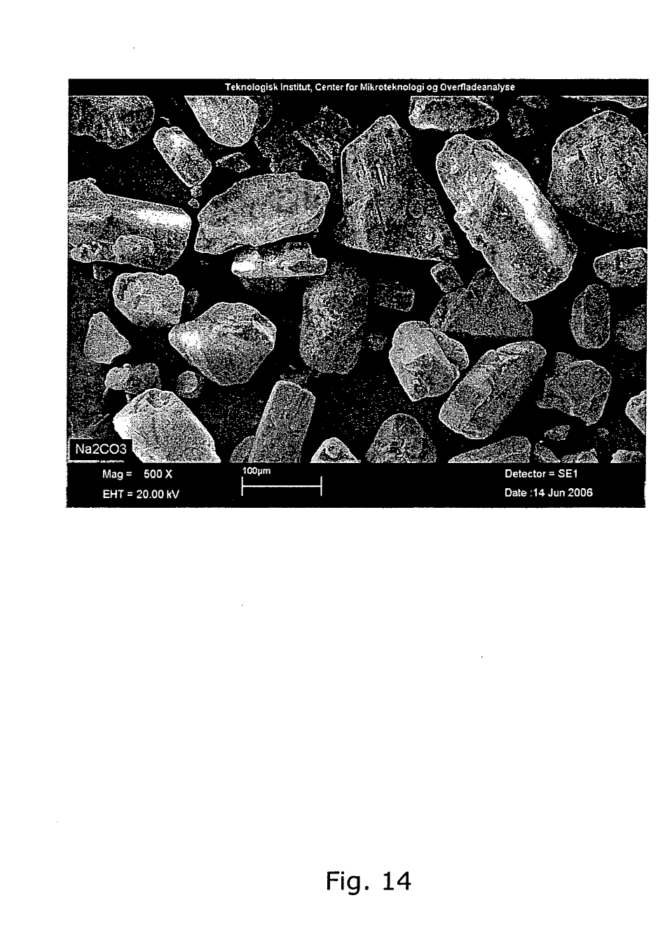

FIG. 13 is a magnification of the particles shown in FIG. 14. The magnification showing the porosity of the particles clearly supports the finding of a high BET area of Sodium Carbonate is illustrated.

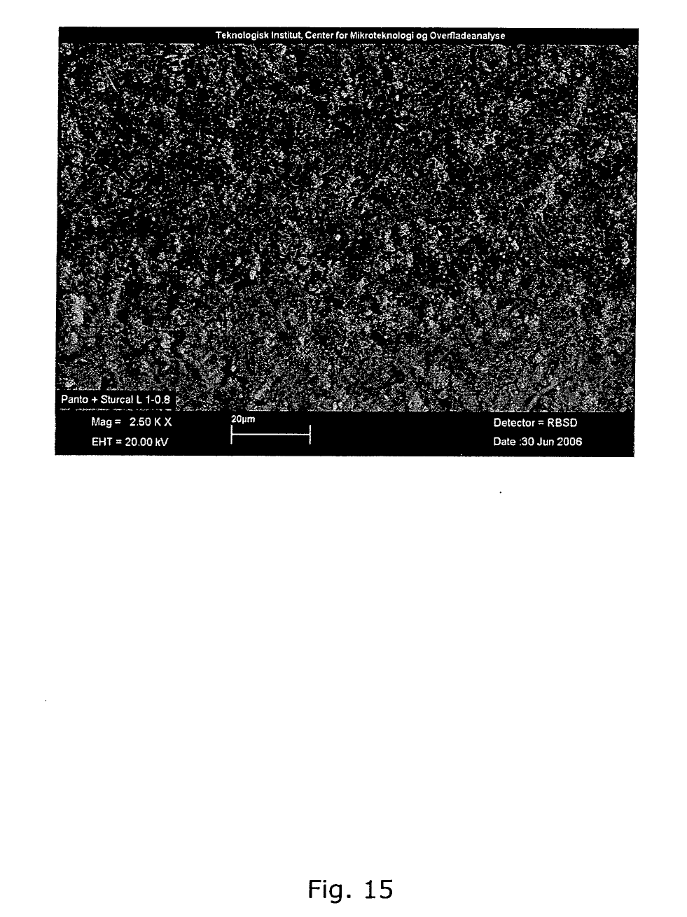

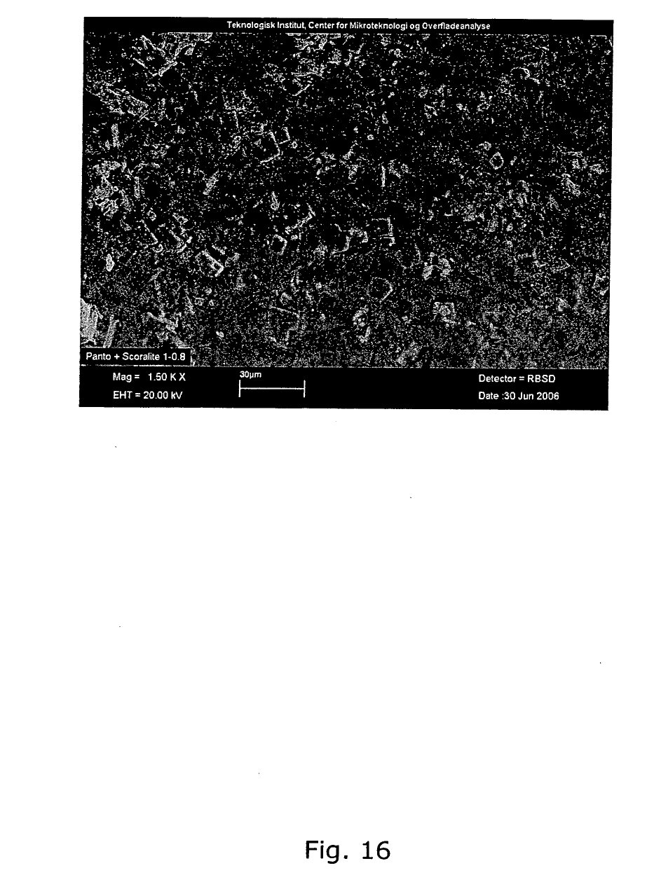

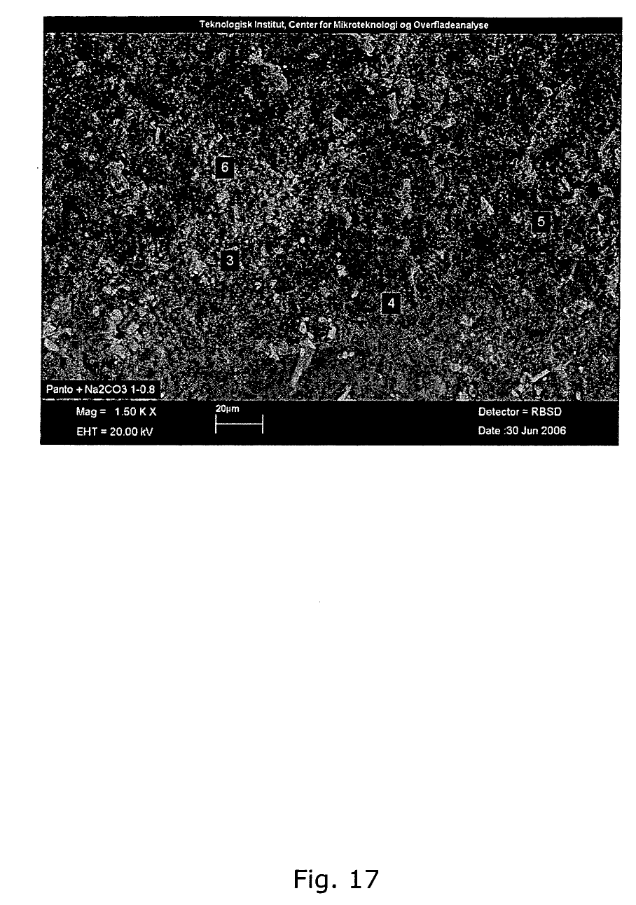

FIGS. 15 and 16 show that the use of Calcium carbonate (Sturcal L) leads to a much more homogeneous mixture than Calcium carbonate (Scoralite). The impact on stability of this difference was illustrated in example 15. FIG. 17 shows that the sodium carbonate particles are crushed during manufacturing. The physical structure of Sturcal L results in an improved distribution of the particles during mechanical pressure. An acceptable stability is obtained as illustrated in examples 15 and 6, 8 and 10.

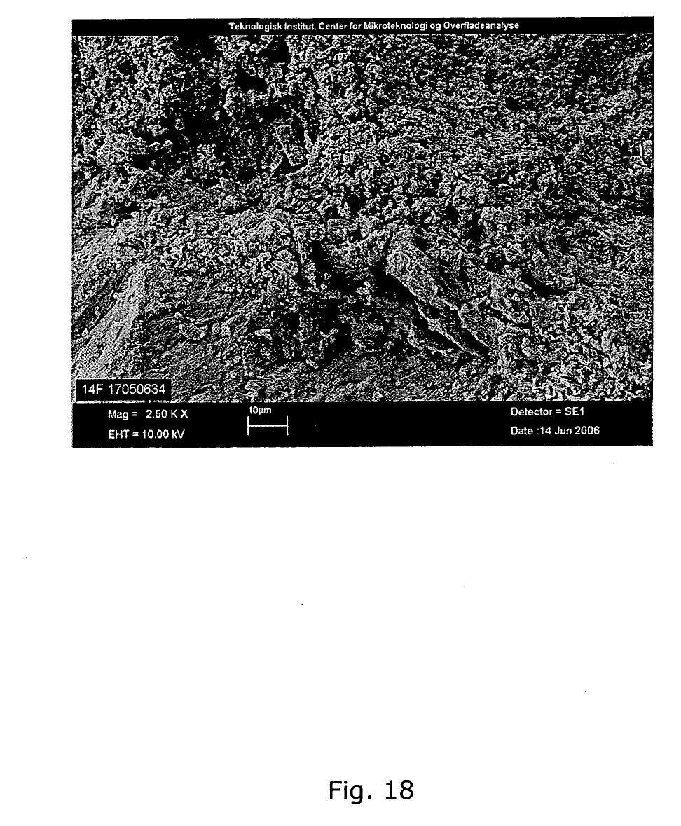

FIG. 18 illustrates the effect of pre-roller compacting pantoprazole prior to mixing with alkaline substance. Slugging (or roller compaction) of the mixture results in “coating” of the surface of the relative large pantoprazole granules with calcium carbonate particles (Sturcal L). This “coating” also leads to an acceptable stability as shown in example 6 and 8. The need for using roller compaction to apply this coat is indicated in example 9, which was based on the use of sodium carbonate.

### Example 18

Impact of disintegrant on dissolution rate.

TABLE 34
Composition of tablet core % w/w:

| | | |
|---|---|---|
| Pantoprazole, 1½ H₂O | 14.1 | 14.1 |
| Calcium carbonate | 11.3 | 11.3 |
| (Sturcal L) | | |
| Pantoprazole:alkaline | 1:0.8 | 1:0.8 |
| excipient | | |
| DiCafos A | 74.1 | 61.1 |
| Crospovidone | — | 13 |
| Mg Stearate | 0.5 | 0.5 |

Based on the tablet core compositions listed above tablets were manufactured as described in example 7 with the following exception that no coat has been applied.

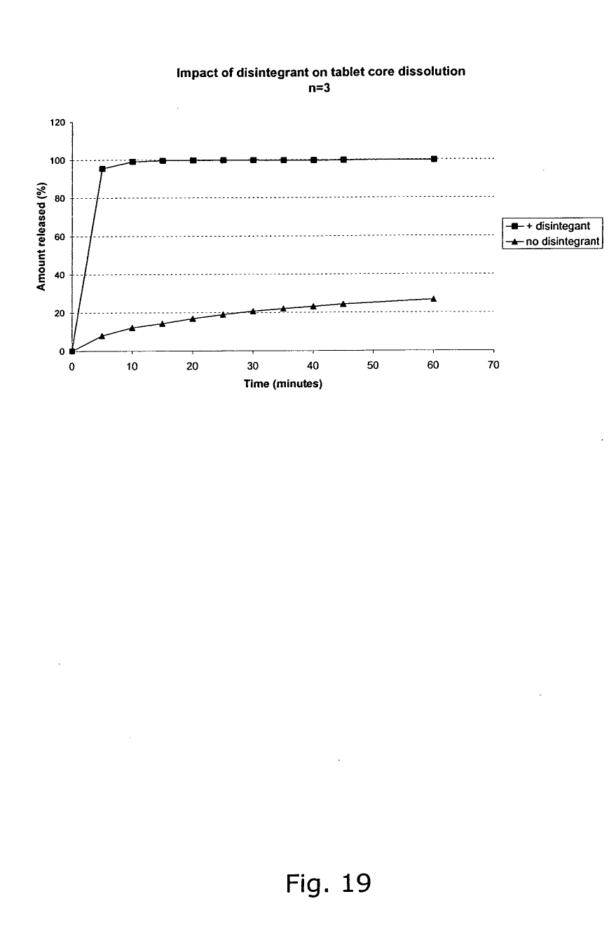

The cores were tested with respect to dissolution rate as described in example 11 with the exception that tablet cores were analysed solely in a dissolution medium with pH 6.8. The results shown in FIG. 19 illustrate the advantages of the incorporation a disintegrant as the presence of a disintegrant in the formulation significantly increases the dissolution rate.

1. A pharmaceutical tablet formulation comprising a benzimidazole as the biologically active component, wherein:
said formulation comprises an enteric coating for protection of the active component from acid attack in the stomach,
said benzimidazole is further stabilized by an alkaline substance in the tablet, wherein said formulation is further characterized by one or more of the following features:
(i) the alkaline substance raw material is an alkali metal carbonate with high water solubility and a BET area of at least about 1 m²/g,
(ii) the alkaline substance raw material is an alkaline earth metal carbonate with low water solubility and a BET area of at least about 1 m²/g,
(iii) the benzimidazole and the alkaline substance raw material have been mixed and dry granulated together prior to dry compression,
(iv) the weight ratio of benzimidazole and alkaline substance is from about 1:0.2-1:5,
(v) the alkaline substance raw material has a pKa of at least about 10 and a BET area of at least about 1 m²/g,
(vi) if the alkaline substance is polyvalent, said alkaline substance has a pKa1-value of 6 or more and a BET area of at least about 1 m²/g,
(vii) the alkaline substance raw material has a BET-area of at least about 1 m²/g,
(viii) the tablet formulation further comprises a disintegrant in an amount of about 1-30% by weight.

2. A method for producing a pharmaceutical tablet formulation comprising a benzimidazole as the biologically active component, wherein:
said formulation comprises an enteric coating for protection of the active component from acid attack in the stomach,
said benzimidazole is further stabilized by an alkaline substance in the tablet,
said method comprising dry granulating steps and dry compressing of tablets, wherein said formulation is further characterized by one or more of the following features:
(i) the alkaline substance is an alkali metal carbonate with high water solubility and a BET area of at least about 1 m²/g prior to any dry granulation and/or dry compression steps,
(ii) the alkaline substance is an alkaline earth metal carbonate with low water solubility and a BET area of at least about 1 m²/g prior to any dry granulation and/or dry compression steps,
(iii) the benzimidazole and the alkaline substance have been mixed and dry granulated together prior to dry compression,
(iv) the weight ratio of benzimidazole and alkaline substance is from about 1:0.2-1:5,
(v) the alkaline substance has a pKa of at least about 10 and a BET area of at least about 1 m²/g prior to any dry granulation and/or dry compression steps,
(vi) if the alkaline substance is polyvalent, said alkaline substance has a pKa1-value of 6 or more and a BET area of at least about 1 m2/g prior to any dry granulation and/or dry compression steps,
(vii) the alkaline substance has a BET-area of at least about 1 m²/g prior to any dry granulation and/or dry compression steps,
(viii) the tablet formulation further comprises a disintegrant in an amount of about 1-30% by weight.

3. A method according to claim 2, wherein the benzimidazole is pantoprazole.

4. A method according to claim 2, wherein the pantoprazole is pantoprazole sodium hydrate or pantoprazole sodium sesquihydrate.

5. A method according to claim 2, wherein said formulation further comprises pharmaceutically acceptable excipients.

6. A method according to claim 2, wherein said formulation comprises crospovidone in an amount of from about 5-15% by weight.

7. A method according to claim 2, wherein said formulation further comprises a subcoat.

8. A method according to claim 2, wherein said formulation is lacking a subcoat.

9. A method according to claim 2, wherein the alkaline substance is a salt of an organic or an inorganic acid and the anion of the salt is carbonate (CO₃²⁻), hydrogenphosphate (HPO₄²⁻) or phosphate (PO₄³⁻).

10. A method according to claim 2, wherein the alkaline substance is a salt of an organic or an inorganic acid and the kation is sodium (Na⁺), calcium (Ca²⁺) or magnesium (Mg²⁺).

11. A method according to claim 2, wherein the salt of the organic and/or inorganic acid according is sodiumcarbonate (Na₂CO₃), trisodiumphosphate (Na₃PO₄), disodiumhydrogenphosphate (Na₂HPO₄), hydrazine or derivatives thereof, lysine or a derivative thereof, arginine or a derivative thereof, or histidine or a derivative thereof.

12. A method according to claim 2, wherein the benzimidazole is omeprazole or a salt and/or a hydrate thereof, lansoprazole or a salt and/or a hydrate thereof, esomeprazol or a salt and/or a hydrate thereof, aripiprazole or a salt and/or a hydrate thereof, rabeprazol or a salt and/or a hydrate thereof, timoprazole or a salt and/or a hydrate thereof.

13. A method according to claim 2, wherein the tablet has a weight in the range of 75 mg to 2.5 g.

14. A method according to claim 2, wherein the dry granulation is provided by means of a roller compactor.

15. A method according to claim 2, wherein the mixture has been subject to sieving prior to tablet compression with a sieve size of 1.25 mm or less.

16. A product obtainable by a method according to claim 2.
